(19) United States
(12) Patent Application Publication
(10) Pub. No.: US 2008/0176947 A1
(43) Pub. Date: Jul. 24, 2008

(54) Heterocyclic-Substituted Alkanamides Useful as Renin Inhibitors

(76) Inventors: Peter Herold, Basel (CH); Robert Mah, Muttenz (CH); Vincenzo Tschinke, Binningen (CH); Stefan Stutz, Basel (CH); Dirk Behnke, Grenzach-Wyhlen (DE); Aleksandar Stojanovic, Basel (CH); Stjepan Jelakovic, Freiburg (DE); Christiane Marti, Rheinfelden (DE)

Correspondence Address: WENDEROTH, LIND & PONACK, L.L.P., 2033 K STREET N. W., SUITE 800, WASHINGTON, DC, 20006-1021, US

(21) Appl. No.: 11/885,967

(22) PCT Filed: Mar. 10, 2006
(86) PCT No.: PCT/EP06/60625

§ 371 Date: Sep. 10, 2007

(30) Foreign Application Priority Data
Mar. 11, 2005 (CH) 0427/05
Sep. 17, 2005 (CH) 1522/05

(51) Int. Cl. A61K 31/165; C07C 237/20; A61P 9/00

(52) U.S. Cl. 514/620; 564/165

(57) ABSTRACT

Compounds of the general formula (I) in which the meanings of the substituents R¹ and R² are as stated in claim 1, have renin-inhibiting properties and can be used as medicines.

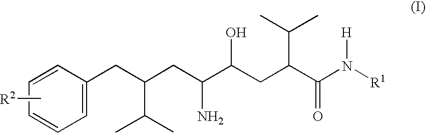

### FIELD OF THE INVENTION

The present invention relates to novel alkanamides, process for their preparation and the use of the compounds as medicines, especially as renin inhibitors.

### BACKGROUND OF THE INVENTION

Alkanamides for use as medicines are disclosed for example in EP 0678503. In relation especially to the renin inhibition, however, there is still a need for active ingredients of high potency. The priority in this is to improve the pharmacokinetic properties. These properties, which aim at better bioavailability, are for example absorption, metabolic stability, solubility or lipophilicity.

### DETAILED DESCRIPTION OF THE INVENTION

The invention therefore provides compounds of the general formula

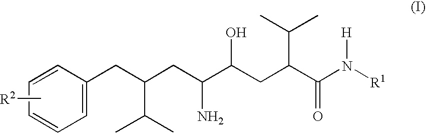

in which

### R¹ is

a) saturated heterocyclyl which is optionally substituted one or more times by C₁₋₈-alkanoyl, C₂₋₆alkenyl, C₂₋₆alkynyl, C₁₋₆alkoxy, C₁₋₆alkoxy-C₁₋₆alkoxy, C₁₋₆alkoxy-C₁₋₆alkyl, C₁₋₆alkoxycarbonylamino, C₁₋₆alkyl, C₀₋₆alkylcarbonylamino, C₁₋₆alkylcarbonyloxy, C₁₋₆alkylenedioxy, optionally N-mono or N,N-di-C₁₋₆-alkylated amino, aryl, optionally N-mono or N,N-di-C₁₋₆-alkylated carbamoyl, optionally esterified carboxy, cyano, C₃₋₈cycloalkoxy, C₃₋₈cycloalkyl-C₀₋₆alkyl, halogen, halo-C₁₋₆alkoxy, halo-C₁₋₆alkyl, heteroaryl, unsaturated, partially saturated or saturated heterocyclyl, hydroxy, nitro, oxide or oxo which is bonded via a C atom; or
b) azepanyl-, azetidinyl-, aziridinyl-, dioxanyl-, dioxepanyl-, dioxolanyl-, dithianyl-, dithiolanyl-, furanyl-, oxathianyl-, oxepanyl-, tetrahydropyranyl-, tetrahydrofuranyl-, tetra-hydrothiophenyl-, tetrahydrothiopyranyl-, thiepanyl- or bicyclic saturated heterocyclyl-C₁₋₄alkyl each of which is optionally substituted one or more times by C₁₋₈alkanoyl, C₂₋₆alkenyl, C₂₋₆alkynyl, C₁₋₆alkoxy, C₁₋₆alkoxy-C₁₋₆alkoxy, C₁₋₆alkoxy-C₁₋₆alkyl, C₁₋₆alkoxycarbonylamino, C₁₋₆alkyl, C₀₋₆alkylcarbonylamino, C₁₋₆alkylcarbonyloxy, C₁₋₆alkylenedioxy, optionally N-mono or N,N-di-C₁₋₆-alkylated amino, aryl, optionally N-mono or N,N-di-C₁₋₆-alkylated carbamoyl, optionally esterified carboxy, cyano, C₃₋₈cycloalkoxy, C₃₋₈cycloalkyl-C₀₋₆alkyl, halogen, halo-C₁₋₆alkoxy, halo-C₁₋₆alkyl, heteroaryl, unsaturated, partially saturated or saturated heterocyclyl, hydroxy, nitro, oxide or oxo; or
c) C₂₋₈alkynyl which is optionally substituted one or more times by C₁₋₈alkanoyl, C₂₋₆alkenyl, C₂₋₆alkynyl, C₁₋₆alkoxy, C₁₋₆alkoxy-C₁₋₆alkoxy, C₁₋₆alkoxy-C₁₋₆alkyl, C₁₋₆alkoxycarbonylamino, C₁₋₆alkyl, C₀₋₆alkylcarbonylamino, C₁₋₆alkylcarbonyloxy, C₁₋₆alkylenedioxy, optionally N-mono or N,N-di-C₁₋₆-alkylated amino, aryl, optionally N-mono or N,N-di-C₁₋₆-alkylated carbamoyl, optionally esterified carboxy, cyano, C₃₋₈cycloalkoxy, C₃₋₈cycloalkyl-C₀₋₆alkyl, halogen, halo-C₁₋₆-alkoxy, halo-C₁₋₆alkyl, heteroaryl, unsaturated, partially saturated or saturated heterocyclyl, hydroxy, nitro or oxo; or
   R² is, independently of one another, 1-4 radicals selected from:
   C₁₋₈alkanoyl, C₁₋₈alkanoyl-C₂₋₄alkoxy bearing the alkanoyl group in a position higher than α, C₁₋₈alkanoylamino-C₁₋₄alkoxy, N—C₁₋₄alkanoylamino-C₁₋₄alkyl, C₁₋₈alkanoyloxy-C₁₋₄alkyl, N′—C₂₋₈alkanoylpiperazino-C₁₋₄alkoxy, N′—C₂₋₈alkanoylpiperazino-C₁₋₄alkyl, C₁₋₈alkanesulphonyl-C₁₋₄alkoxy, C₁₋₈alkanesulphonyl-C₁₋₄alkyl, C₁₋₈alkanesulphonylamino-C₁₋₄alkoxy, C₁₋₄alkanesulphonylamino-C₁₋₄alkyl, C₁₋₈alkanesulphonyl-C₁₋₄(hydroxy)alkoxy, C₂₋₈alkenyloxy, C₂₋₈alkenyloxy-C₁₋₄alkoxy, C₂₋₈alkenyloxy-C₁₋₄alkyl, C₁₋₈alkoxy, C₁₋₆alkoxy-C₁₋₆alkanoyl, C₁₋₄alkoxy-C₂₋₄alkenyl, C₁₋₄alkoxy-C₂₋₄alkenyloxy, C₁₋₄alkoxy-C₁₋₄alkoxy, C₁₋₄alkoxy-C₁₋₄alkoxy-C₁₋₄alkyl, C₁₋₄alkoxy-C₁₋₄alkyl, C₁₋₄alkoxycarbonyl-C₁₋₄alkoxy, C₁₋₄alkoxycarbonyl-C₁₋₄alkyl, C₁₋₈alkoxycarbonylamino-C₁₋₄alkoxy, C₁₋₈alkoxycarbonylamino-C₁₋₄alkyl, C₁₋₈alkyl, C₁₋₄alkylamino, N,N-di-C₁₋₄alkylamino, C₁₋₄alkylamino-C₁₋₄alkoxy, N,N-di-C₁₋₄alkylamino-C₁₋₄alkoxy, C₁₋₄alkylamino-C₁₋₄alkyl, N,N-di-C₁₋₄alkylamino-C₁₋₄alkyl, N-mono- or N,N-di-C₁₋₄alkylcarbamoyl-C₁₋₄alkoxy, N-mono- or N,N-di-C₁₋₄alkylcarbamoyl-C₁₋₄alkyl, N′—C₁₋₄alkylpiperazino-C₁₋₄alkoxy, N′—C₁₋₄alkylpiperazino-C₁₋₄alkyl, C₁₋₄alkylthio-C₁₋₄alkoxy, C₁₋₄alkylthio-C₁₋₄(hydroxy)alkoxy, C₁₋₄alkylthio-C₁₋₄alkyl, amino-C₂₋₄alkoxy, amino-C₁₋₄alkyl, carbamoyl-C₁₋₄alkoxy, carbamoyl-C₁₋₈alkyl, carboxy-C₁₋₄alkoxy, carboxy-C₁₋₄alkyl, cyano-C₁₋₄alkoxy, cyano-C₁₋₄alkyl, C₃₋₈-cycloalkoxy, C₃₋₈cycloalkoxy-C₁₋₄alkoxy, C₃₋₈cycloalkoxy-C₁₋₄alkyl, C₃₋₈cycloalkyl, S,S-dioxothiomorpholino-C₁₋₄alkoxy, S,S-dioxothiomorpholino-C₁₋₄alkyl, halogen, halo-C₁₋₄alkoxy, halo-C₁₋₄alkyl, halo-C₂₋₈(hydroxy)alkoxy, hydroxy, hydroxy-C₂₋₈alkoxy, hydroxy-C₂₋₈alkyl, imidazolylthio-C₁₋₄alkoxy, imidazolylthio-C₁₋₄alkyl, optionally N-oxidized morpholino-C₁₋₄alkoxy, morpholino-C₁₋₄alkyl, S-oxothiomorpholino-C₁₋₄alkoxy, S-oxothiomorpholino-C₁₋₄alkyl, piperazino-C₁₋₄alkoxy, piperazino-C₁₋₄alkyl, piperidino-C₁₋₄alkoxy, piperidino-C₁₋₄alkyl, optionally partially hydrogenated pyridyl- or N-oxidopyridyl-C₁₋₄alkoxy, optionally partially hydrogenated pyridyl- or N-oxidopyridyl-C₁₋₄alkyl, optionally N-oxidized pyridylthio-C₁₋₄alkoxy, optionally N-oxidized pyridylthio-C₁₋₄alkyl, pyrimidinylthio-C₁₋₄alkoxy, pyrimidinylthio-C₁₋₄alkyl, pyrrolidino-C₁₋₄alkoxy, pyrrolidino-C₁₋₄alkyl, thiazolinylthio-C₁₋₄alkoxy, thiazolinylthio-C₁₋₄alkyl, thiazolyl-C₁₋₄alkoxy, thiazolylthio-C₁₋₄alkoxy, thiazolylthio-C₁₋₄alkyl, thiomorpholino-C₁₋₄alkoxy, thiomorpholino-C₁₋₄alkyl, trifluoro-C₁₋₈alkanesulphonyl-C₁₋₄alkoxy, trifluoro-C₁₋₈alkanesulphonylamino-C₁₋₄alkyl, phenyl or naphthyl which is unsubstituted or mono-, di- or trisubstituted by C₁₋₄alkoxy, C₁₋₄alkyl, C₁₋₄alkylamino, di-C₁₋₄alkylamino, halogen, hydroxy and/or trifluoromethyl, and phenyl- or naphthyl-C₁₋₄alkoxy which is unsubstituted or mono-, di- or tri-substituted by C₁₋₄alkoxy, C₁₋₄alkyl, C₁₋₄alkylamino, di-C₁₋₄alkylamino, halogen, hydroxy and/or trifluoromethyl,
   and salts, especially pharmaceutically acceptable salts thereof;
   excepting compounds in which R¹ is optionally substituted N-acetylpiperidin-4-yl or optionally substituted oxopiperidinyl.

The term saturated heterocyclyl refers to 3-16-membered, mono- or bicyclic saturated heterocyclic radicals having 1 to 4 nitrogen and/or 1 or 2 sulphur or oxygen atoms. Preferred radicals have 3-8 members, particularly preferably 5 or 6 members, and are monocyclic and are optionally fused to a 3-8-membered ring which may be carbocyclic or heterocyclic. A further preferred group of heterocyclic radicals are bicyclic heterocycles which have a spirocyclic or bridged ring. Preferred heterocyclic radicals have in each ring 1 nitrogen, oxygen or sulphur atom, 1-2 nitrogen atoms and 1-2 oxygen atoms or 1-2 nitrogen atoms and 1-2 sulphur atoms, with at least one, preferably 1-7, carbon atom(s) being present in each ring. Examples of heterocyclyl radicals are azepanyl, azetidinyl, aziridinyl, dioxanyl, [1,4]dioxepanyl, dioxolanyl, dithianyl, dithiolanyl, morpholinyl, oxathianyl, oxepanyl, piperazinyl, piperidinyl, pyrrolidinyl, tetrahydropyranyl, tetrahydrofuranyl, tetrahydrothiophenyl, tetrahydrothiopyranyl, thiepanyl or thiomorpholinyl. Examples of bicyclic heterocyclyl radicals are 2,5-dioxabicyclo[4.1.0]heptanyl, 2-oxabicyclo[2.2.1]heptanyl, 2-oxabicyclo[4.1.0]heptanyl, 3-oxabicyclo[4.1.0]heptanyl, 7-oxabicyclo[2.2.1]heptanyl, 2-oxabicyclo[3.1.0]hexanyl, 3-oxabicyclo[3.1.0]hexanyl, 1-oxaspiro[2.5]octanyl, 6-oxaspiro[2.5]octanyl or 3-oxabicyclo[3.3.1]nonanyl.

Heterocyclyl may be unsubstituted or substituted one or more times, e.g. once or twice, by C₁₋₈alkanoyl, C₂₋₆alkenyl, C₂₋₆alkynyl, C₁₋₆alkoxy, C₁₋₆alkoxy-C₁₋₆alkoxy, C₁₋₆alkoxy-C₁₋₆alkyl, C₁₋₆alkoxycarbonylamino, C₁₋₆alkyl, C₀₋₆alkylcarbonylamino, C₁₋₆alkylcarbonyloxy, C₁₋₆alkylenedioxy, optionally N-mono or N,N-di-C₁₋₆-alkylated amino, aryl, optionally N-mono or N,N-di-C₁₋₆-alkylated carbamoyl, optionally esterified carboxy, cyano, C₃₋₈cycloalkoxy, C₃₋₈cycloalkyl-C₀₋₆alkyl, halogen, halo-C₁₋₆alkoxy, halo-C₁₋₆alkyl, heteroaryl, heterocyclyl, hydroxy, nitro, oxide or oxo.

C₁₋₈Alkanoyl is, for example, formyl, ethanoyl, propanoyl or butanoyl.

C₁₋₆Alkyl may be straight-chain or branched and/or bridged and is for example methyl, ethyl, propyl, isopropyl, butyl, isobutyl, secondary butyl, tertiary butyl or a pentyl or hexyl group.

C₁₋₆Alkylamino is, for example, methylamino, ethylamino, propylamino or butylamino.

Di-C₁₋₆alkylamino is, for example, dimethylamino, N-methyl-N-ethylamino, diethylamino, N-methyl-N-propylamino or N-butyl-N-methylamino.

C₂₋₆Alkenyl may be straight-chain or branched and is, for example, allyl or vinyl

C₂₋₆Alkynyl may be straight-chain or branched and is, for example, ethynyl.

C₁₋₆Alkoxy is, for example, methoxy, ethoxy, propyloxy, isopropyloxy, butyloxy, isobutyloxy, secondary butyloxy, tertiary butyloxy, pentyloxy or hexyloxy.

C₁₋₆Alkoxycarbonylamino is preferably C₂-C₅alkoxycarbonylamino such as ethoxycarbonylamino, propyloxycarbonylamino, isopropyloxycarbonylamino, butyloxycarbonylamino, isobutyloxycarbonylamino, secondary butyloxycarbonylamino or tertiary butyloxycarbonylamino.

C₁₋₆Alkylcarbonyloxy is, for example, acetyloxy, propionyloxy, propylcarbonyloxy, isopropylcarbonyloxy, butylcarbonyloxy, isobutylcarbonyloxy, secondary butylcarbonyloxy, tertiary butylcarbonyloxy, pentylcarbonyloxy or hexylcarbonyloxy.

C₀₋₆Alkylcarbonylamino is, for example, formylamino, acetylamino, propionylamino, propylcarbonylamino, isopropylcarbonylamino, butylcarbonylamino, isobutylcarbonylamino, secondary butylcarbonylamino, tertiary butylcarbonylamino, pentylcarbonylamino or hexylcarbonylamino.

Halogen is, for example, fluorine, chlorine, bromine or iodine, preferably fluorine or chlorine.

Halo-C₁₋₆alkoxy is, for example, alkoxy substituted one or more times by fluorine, chlorine, bromine or iodine, including mixed, e.g. fluorine and chlorine, substitutions, with preference for perfluorinated radicals such as trifluoromethoxy.

Halo-C₁₋₆alkyl is, for example, alkyl substituted one or more times by fluorine, chlorine, bromine or iodine, including mixed, e.g. fluorine and chlorine, substitutions, with preference for perfluorinated radicals such as trifluoromethyl.

C₁₋₆Alkylenedioxy is, for example, methylenedioxy, ethylenedioxy, 1,3-propylenedioxy or 1,2-propylenedioxy.

Optionally N-mono or N,N-di-C₁₋₈-alkylated carbamoyl is, for example, carbamoyl, methylcarbamoyl, ethylcarbamoyl, N,N-dimethylcarbamoyl, N,N-diethylcarbamoyl or propylcarbamoyl.

Optionally esterified carboxy is, for example, carboxy esterified with C₀₋₆alkyl, such as carboxy or C₁₋₆alkoxycarbonyl.

C₃₋₈Cycloalkoxy is preferably 3-, 5- or 6-membered cycloalkoxy such as cyclopropyloxy, cyclopentyloxy and cyclohexyloxy.

C₃₋₈Cycloalkyl-C₀₋₆alkyl is preferably 3-, 5- or 6-membered cycloalkyl such as cyclopropyl, cyclopropylmethyl, cyclopentyl and cyclohexyl.

Cyano-C₁₋₄alkoxy is, for example, cyanomethoxy, 2-cyanoethoxy, 2- or 3-cyanopropyloxy or 4-cyanobutyloxy, especially cyanomethoxy.

Cyano-C₁₋₄alkyl is, for example, cyanomethyl, 2-cyanoethyl, 2- or 3-cyanopropyl, 2-cyano-2-methylpropyl or 4-cyanobutyl, especially cyanomethyl.

N,N-Di-C₁₋₄alkylamino is, for example, dimethylamino, N-methyl-N-ethylamino, diethylamino, N-methyl-N-propylamino or N-butyl-N-methylamino.

N,N-Di-C₁₋₄alkylamino-C₁₋₄alkoxy is 2-dimethylaminoethoxy, 3-dimethylaminopropyloxy, 4-dimethylaminobutyloxy, 2-diethylaminoethoxy, 2-(N-methyl-N-ethylamino)ethoxy or 2-(N-butyl-N-methylamino)ethoxy.

N,N-Di-C₁₋₄alkylamino-C₁₋₄alkyl is, for example, 2-dimethylaminoethyl, 3-dimethylaminopropyl, 4-dimethylaminobutyl, 2-diethylaminoethyl, 2-(N-methyl-N-ethylamino)ethyl or 2-(N-butyl-N-methylamino)ethyl.

N,N-Di-C₁₋₄alkylcarbamoyl-C₁₋₄alkoxy is, for example, methyl- or dimethylcarbamoyl-C₁₋₄alkoxy such as N-methyl-, N-butyl- or N,N-dimethylcarbamoylmethoxy, 2-(N-methylcarbamoyl)ethoxy, 2-(N-butylcarbamoyl)ethoxy, 2-(N,N-dimethylcarbamoyl)ethoxy, 3-(N-methylcarbamoyl)propyloxy, 3-(N-butylcarbamoyl)propyloxy, 3-(N,N-dimethylcarbamoyl)propyloxy or 4-(N-methylcarbamoyl)butyloxy, 4-(N-butylcarbamoyl)butyloxy or 4-(N,N-dimethylcarbamoyl)butyloxy, especially N-methyl-, N-butyl- or N,N-dimethylcarbamoylmethoxy.

N,N-Di-C₁₋₄alkylcarbamoyl-C₁₋₄alkyl is, for example, 2-dimethylcarbamoylethyl, 3-dimethylcarbamoylpropyl, 2-dimethylcarbamoylpropyl, 2-(dimethylcarbamoyl)-2-methylpropyl or 2-(dimethylcarbamoyl)butyl.

Optionally partially hydrogenated pyridyl- or N-oxidopyridyl-C₁₋₄alkoxy is, for example, pyridyl- or N-oxidopyridylmethoxy, 2-pyridylethoxy, 2- or 3-pyridylpropyloxy or 4-pyridylbutyloxy, especially 3- or 4-pyridylmethoxy.

Optionally partially hydrogenated pyridyl- or N-oxidopyridyl-C₁₋₄alkyl is, for example, pyridyl- or N-oxidopyridylmethyl, 2-pyridylethyl, 2- or 3-pyridylpropyl or 4-pyridylbutyl, especially 3- or 4-pyridylmethyl.

Morpholino-C₁₋₄alkoxy may be N-oxidized and is, for example, 1-morpholinoethoxy, 3-morpholinopropyloxy or 1-(morpholino-2-methyl)propyloxy.

Morpholino-C₁₋₄alkyl may be N-oxidized and is, for example, morpholinomethyl, 2-morpholinoethyl, 3-morpholinopropyl or 1- or 2-(4-morpholino)butyl.

Piperazino-C₁₋₄alkyl is, for example, piperazinomethyl, 2-piperazinoethyl or 3-piperazinopropyl.

Piperidino-C₁₋₄alkoxy is, for example, piperidinomethoxy, 2-piperidinoethoxy or 3-piperidinopropyloxy.

Piperidino-C₁₋₄alkyl is, for example, piperidinomethyl, 2-piperidinoethyl or 3-piperidinopropyl.

Pyrrolidino-C₂₋₄alkoxy is, for example, 2-pyrrolidinoethoxy or 3-pyrrolidinopropyloxy.

Pyrrolidino-C₁₋₄alkyl is, for example, pyrrolidinomethyl, 2-pyrrolidinoethyl or 3-pyrrolidinopropyl.

S-Oxothiomorpholino-C₁₋₄alkyl is, for example, S-oxothiomorpholinomethyl or 2-(S-oxothiomorpholino)ethyl.

Thiazolyl-C₁₋₄alkoxy is, for example, thiazolylmethoxy, 2-thiazolylethoxy or 3-thiazolylpropyloxy.

Thiomorpholino-C₁₋₄alkyl or S,S-dioxothiomorpholino-C₁₋₄alkyl is, for example, thiomorpholino-C₁₋₄alkyl such as -methyl or -ethyl, or S,S-dioxothiomorpholino-C₁₋₄alkyl such as -methyl or -ethyl.

Depending on the presence of asymmetric carbon atoms, the compounds of the invention may be in the form of mixtures of isomers, specifically as racemates, or in the form of pure isomers, specifically of optical antipodes. The invention includes all these forms. Mixtures of diastereomers, diastereomeric racemates or mixtures of diastereomeric racemates can be fractionated by conventional methods, e.g. by column chromatography, thin-layer chromatography, HPLC and the like.

Salts of compounds with salt-forming groups are in particular acid addition salts, salts with bases or, if a plurality of salt-forming groups is present, optionally also mixed salts or inner salts.

Salts are primarily the pharmaceutically acceptable or non-toxic salts of compounds of the formula (I). Such salts are formed for example by compounds of the formula (I) having an acidic group, e.g. a carboxy or sulpho group, and are for example their salts with suitable bases, such as non-toxic metal salts derived from metals of group Ia, Ib, IIa and IIb of the Periodic Table of the Elements, e.g. alkali metal, in particular lithium, sodium or potassium, salts, alkaline earth metal salts, for example magnesium or calcium salts, furthermore zinc salts or ammonium salts, also salts formed with organic amines such as optionally hydroxy-substituted mono-, di- or trialkylamines, especially mono-, di- or tri-lower-alkylamines, or with quaternary ammonium bases, e.g. methyl-, ethyl-, diethyl- or triethylamine, mono-, bis- or tris(2-hydroxy-lower-alkyl)amines such as ethanol-, diethanol- or triethanolamine, tris(hydroxymethyl)methylamine or 2-hydroxy-tertiary-butylamine, N,N-di-lower-alkyl-N-(hydroxy-lower-alkyl)amine, such as N,N-dimethyl-N-(2-hydroxyethyl)amine, or N-methyl-D-glucamine, or quaternary ammonium hydroxides such as tetrabutylammonium hydroxide. The compounds of the formula I having a basic group, e.g. an amino group, can form acid addition salts, e.g. with suitable inorganic acids, e.g. hydrohalic acid such as hydrochloric acid, hydrobromic acid, sulphuric acid with replacement of one or both protons, phosphoric acid with replacement of one or more protons, e.g. orthophosphoric acid or metaphosphoric acid, or pyrophosphoric acid with replacement of one or more protons, or with organic carboxylic, sulphonic or phosphonic acids or N-substituted sulphamic acids, e.g. acetic acid, propionic acid, glycolic acid, succinic acid, maleic acid, hydroxymaleic acid, methylmaleic acid, fumaric acid, malic acid, tartaric acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid, isonicotinic acid, furthermore amino acids such as, for example, the α-amino acids mentioned hereinbelow, and methanesulphonic acid, ethanesulphonic acid, 2-hydroxyethanesulphonic acid, ethane-1,2-disulphonic acid, benzenesulphonic acid, 4-toluenesulphonic acid, naphthalene-2-sulphonic acid, 2- or 3-phosphoglycerate, glucose 6-phosphate, N-cyclohexylsulphamic acid (to form cyclamates) or with other acidic organic compounds such as ascorbic acid. Compounds of the formula (I) having acidic and basic groups may also form inner salts.

Pharmaceutically unsuitable salts may also be used for isolation and purification.

The compounds of the formula (I) also include compounds in which one or more atoms are replaced by their stable, non-radioactive isotopes; for example a hydrogen atom by deuterium.

Prodrug derivatives of the compounds described herein are derivatives thereof which on in vivo use liberate the original compound by a chemical or physiological process. A prodrug may for example be converted into the original compound when a physiological pH is reached or by enzymatic conversion. Possible examples of prodrug derivatives are esters of freely available carboxylic acids, S- and O-acyl derivatives of thiols, alcohols or phenols, the acyl group being defined as above. Preferred derivatives are pharmaceutically acceptable ester derivatives which are converted by solvolysis in physiological medium into the original carboxylic acid, such as, for example, lower alkyl esters, cycloalkyl esters, lower alkenyl esters, benzyl esters, mono- or disubstituted lower alkyl esters such as lower ω-(amino, mono- or dialkylamino, carboxy, lower alkoxycarbonyl)-alkyl esters or such as lower α-(alkanoyloxy, alkoxycarbonyl or dialkylaminocarbonyl)-alkyl esters; conventionally, pivaloyloxymethyl esters and similar esters are used as such.

Because of the close relationship between a free compound, a prodrug derivative and a salt compound, a particular compound in this invention also includes its prodrug derivative and salt form, where this is possible and appropriate.

The groups of compounds mentioned hereinafter are not to be regarded as closed; on the contrary, it is possible for parts of these groups of compounds to be interchanged or replaced by the definitions given above, or omitted, in a worthwhile manner, e.g. to replace general by more specific definitions.

Further preferred compounds have the general formula

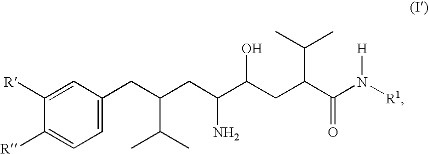

in which
R¹ has the meaning indicated for the compound of the formula (I) and
R′ and R″, independently of one another, have the meanings indicated for R² for the compound of the formula (I).

R² is preferably, independently of one another, 1-4 radicals selected from: C₁₋₈alkanoylamino-C₁₋₄alkoxy, N—C₁₋₄alkanoylamino-C₁₋₄alkyl, C₁₋₈alkoxy, C₁₋₄alkoxy-C₁₋₄alkoxy, C₁₋₄alkoxy-C₁₋₄alkoxy-C₁₋₄alkyl, C₁₋₄alkoxy-C₁₋₄alkyl, C₁₋₈alkoxycarbonylamino-C₁₋₄alkoxy, C₁₋₈alkoxycarbonylamino-C₁₋₄alkyl, C₁₋₈alkyl, halogen, trifluoromethoxy and trifluoromethyl. R² is particularly preferably, independently of one another, 1-4 radicals selected from: C₁₋₈alkoxy, C₁₋₄alkoxy-C₁₋₄alkoxy, C₁₋₄alkoxy-C₁₋₄alkoxy-C₁₋₄alkyl, C₁₋₄alkoxy-C₁₋₄alkyl, C₁₋₈alkyl, halogen, trifluoromethoxy and trifluoromethyl.

R¹ is preferably optionally substituted C₂₋₆alkynyl, optionally substituted saturated heterocyclyl bonded via a C atom or optionally substituted saturated bicyclic heterocyclyl-C₀₋₄alkyl. R¹ is particularly preferably optionally substituted C₂₋₆alkynyl, optionally substituted saturated bicyclic heterocyclyl-C₀₋₄alkyl, optionally substituted pyrrolidinyl, C₁₋₆-alkylated or C₃₋₈-cycloalkyl-C₀₋₆-alkylated piperidine, optionally substituted tetrahydrofuranyl, optionally substituted tetrahydrofuranylmethyl, optionally substituted tetrahydropyranyl or optionally substituted tetrahydropyranylmethyl.

R¹ is particularly preferably also optionally substituted tetrahydrofuranylmethyl, optionally substituted tetrahydropyranylmethyl, optionally substituted saturated heterocyclyl which is bonded via a C atom or optionally substituted saturated bicyclic heterocyclyl-C₀₋₄alkyl, where the heterocyclyl radical in each case comprises an oxygen atom as heteroatom.

Also particularly preferred are compounds of the general formula (I′) where

R′ is C₁₋₈alkanoylamino-C₁₋₄alkoxy, N—C₁₋₄alkanoylamino-C₁₋₄alkyl, C₁₋₄alkoxy-C₁₋₄alkoxy-C₁₋₄alkyl, C₁₋₄alkoxy-C₁₋₄alkoxy, C₁₋₄alkoxy-C₁₋₄alkyl, C₁₋₈alkoxycarbonylamino-C₁₋₄alkoxy or C₁₋₈alkoxycarbonylamino-C₁₋₄alkyl; and
R″ is C₁₋₈alkanoyl, C₁₋₈alkoxy, C₁₋₈alkyl, difluoroethyl, halogen, trifluoromethoxy or trifluoromethyl.

Also particularly preferred are compounds of the general formula (I′) where

R′ is C₁₋₄alkoxy-C₁₋₄alkoxy;
R″ is fluorine; and
R¹ is optionally substituted tetrahydrofuranylmethyl, optionally substituted tetrahydropyranylmethyl, optionally substituted saturated heterocyclyl which is bonded via a C atom or optionally substituted saturated bicyclic heterocyclyl-C₀₋₄alkyl, where the heterocyclyl radical preferably in each case comprises an oxygen atom as heteroatom.

Particularly preferred in each case are those compounds of the formula I, in which at least one, for example one, two, three or, preferably, all four asymmetric C atoms of the main chain have the stereochemistry shown in formula IA (“S” in each case), where the substituents each have the meanings indicated above, and their pharmaceutically acceptable salts.

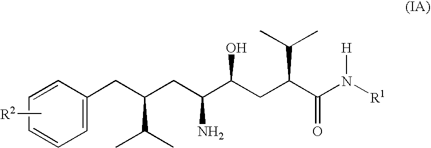

The compounds of the formula (I) and formula (IA) can be prepared in an analogous manner to the preparation process disclosed in the literature (see WO 2002008172 and WO 2002002508 and literature cited therein) (scheme). Further preparation processes are described for example in EP 678503, WO 01/09079, WO 01/09083, WO 02/02487, WO 02/02500, WO 02/092828 and in Helvetica Chemica Acta 86 (2003), 2848-2870 and literature cited therein.

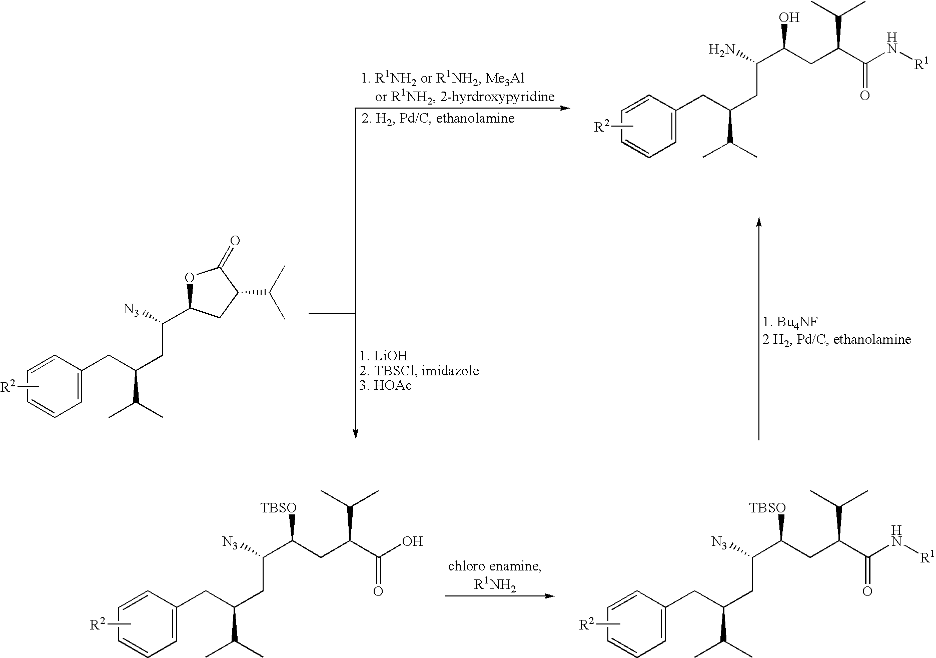

Details of the specific preparation variants can be found in the examples.

The compounds of the formula (I) can also be prepared in optically pure form. Separation into antipodes can take place by methods known per se, either preferably at an early stage in the synthesis by salt formation with an optically active acid such as, for example, (+)- or (−)-mandelic acid and separation of the diastereomeric salts by fractional crystallization or preferably at a rather late stage by derivatizing with a chiral auxiliary component such as, for example, (+)- or (−)-camphanoyl chloride, and separation of the diastereomeric products by chromatography and/or crystallization and subsequent cleavage of the linkage to the chiral auxiliary. The pure diastereomeric salts and derivatives can be analysed to determine the absolute configuration of the contained piperidine by conventional spectroscopic methods, with X-ray spectroscopy on single crystals representing a particularly suitable method.

The compounds of the formula (I), and of the formula (IA), and their pharmaceutically acceptable salts have an inhibitory effect on the natural enzyme renin. The latter passes from the kidneys into the blood and there brings about the cleavage of angiotensinogen to form the decapeptide angiotensin I which is then cleaved in the lung, the kidneys and other organs to the octapeptide angiotensin II. Angiotensin II raises the blood pressure both directly by arterial constriction, and indirectly by releasing the hormone aldosterone, which retains sodium ions, from the adrenals, which is associated with an increase in the extracellular fluid volume. This increase is attributable to the effect of angiotensin II itself or of the heptapeptide angiotensin III formed therefrom as cleavage product. Inhibitors of the enzymatic activity of renin bring about a reduction in the formation of angiotensin I and, as a consequence thereof, the formation of a smaller amount of angiotensin II. The reduced concentration of this active peptide hormone is the direct cause of the blood pressure-lowering effect of renin inhibitors.

The effect of renin inhibitors is detected inter alia experimentally by means of in vitro tests where the reduction in the formation of angiotensin I is measured in various systems (human plasma, purified human renin together with synthetic or natural renin substrate). The following in vitro test of Nussberger et al. (1987) J. Cardiovascular Pharmacol., Vol. 9, pp. 39-44, is used inter alia. This test measures the formation of angiotensin I in human plasma. The amount of angiotensin I formed is determined in a subsequent radioimmunoassay. The effect of inhibitors on the formation of angiotensin I is tested in this system by adding various concentrations of these substances. The IC₅₀ is defined as the concentration of the particular inhibitor which reduces the formation of angiotensin I by 50%. The compounds of the present invention show inhibitory effects in the in vitro systems at minimal concentrations of about 10⁻⁶ to about 10⁻¹⁰ mol/l.

Renin inhibitors bring about a fall in blood pressure in salt-depleted animals. Human renin differs from renin of other species. Inhibitors of human renin are tested using primates (marmosets, Callithrix jacchus) because human renin and primate renin are substantially homologous in the enzymatically active region. The following in vivo test is employed inter alia: the test compounds are tested on normotensive marmosets of both sexes with a body weight of about 350 g, which are conscious, unrestrained and in their normal cages. Blood pressure and heart rate are measured with a catheter in the descending aorta and are recorded radiometrically. Endogenous release of renin is stimulated by combining a low-salt diet for 1 week with a single intramuscular injection of furosemide (5-(aminosulphonyl)-4-chloro-2-[(2-furanylmethyl)amino]benzoic acid) (5 mg/kg). 16 hours after the furosemide injection, the test substances are administered either directly into the femoral artery by means of a hypodermic needle or as suspension or solution by gavage into the stomach, and their effect on blood pressure and heart rate is evaluated. The compounds of the present invention have a blood pressure-lowering effect in the described in vivo test with i.v. doses of about 0.003 to about 0.3 mg/kg and with oral doses of about 0.3 to about 30 mg/kg.

The compounds of the formula (I), and preferably of the formula (IA), and their pharmaceutically acceptable salts can be used as medicines, e.g. in the form of pharmaceutical products. The pharmaceutical products can be administered enterally, such as orally, e.g. in the form of tablets, lacquered tablets, sugar-coated tablets, hard and soft gelatine capsules, solutions, emulsions or suspensions, nasally, e.g. in the form of nasal sprays, rectally, e.g. in the form of suppositories, or transdermally, e.g. in the form of ointments or patches. However, administration is also possible parenterally, such as intramuscularly or intravenously, e.g. in the form of solutions for injection.

Tablets, lacquered tablets, sugar-coated tablets and hard gelatine capsules can be produced by processing the compounds of the formula (I), or preferably of the formula (IA), and their pharmaceutically acceptable salts with pharmaceutically inert inorganic or organic excipients. Excipients of these types which can be used for example for tablets, sugar-coated tablets and hard gelatine capsules are lactose, maize starch or derivatives thereof, talc, stearic acid or salts thereof etc.

Excipients suitable for soft gelatine capsules are, for example, vegetable oils, waxes, fats, semisolid and liquid polyols etc.

Excipients suitable for producing solutions and syrups are, for example, water, polyols, sucrose, invert sugar, glucose etc.

Excipients suitable for solutions for injection are, for example, water, alcohols, polyols, glycerol, vegetable oils, bile acids, lecithin etc.

Excipients suitable for suppositories are, for example, natural or hardened oils, waxes, fats, semiliquid or liquid polyols etc.

The pharmaceutical products may in addition comprise preservatives, solubilizers, viscosity-increasing substances, stabilizers, wetting agents, emulsifiers, sweeteners, colorants, aromatizers, salts to alter the osmotic pressure, buffers, coating agents or antioxidants. They may also comprise other substances of therapeutic value.

The present invention further provides the use of the compounds of the formula (I), or preferably of the formula (IA), and their pharmaceutically acceptable salts in the treatment or prevention of high blood pressure, heart failure, glaucoma, myocardial infarction, renal failure restenoses and stroke.

The compounds of the formula (I), and preferably of the formula (IA), and their pharmaceutically acceptable salts can also be administered in combination with one or more agents having cardiovascular activity, e.g. α- and β-blockers such as phentolamine, phenoxybenzamine, prazosin, terazosin, tolazine, atenolol, metoprolol, nadolol, propranolol, timolol, carteolol etc.; vasodilators such as hydralazine, minoxidil, diazoxide, nitroprusside, flosequinan etc.; calcium antagonists such as aminone, bencyclan, diltiazem, fendiline, flunarizine, nicardipine, nimodipine, perhexyline, verapamil, gallopamil, nifedipine etc.; ACE inhibitors such as cilazapril, captopril, enalapril, lisinopril etc.; potassium activators such as pinacidil; antiserotoninergics such as ketanserine; thromboxane synthetase inhibitors; neutral endopeptidase inhibitors (NEP inhibitors); angiotensin II antagonists; and diuretics such as hydrochlorothiazide, chlorothiazide, acetazolamide, amiloride, bumetanide, benzthiazide, ethacrynic acid, furosemide, indacrinone, metolazone, spironolactone, triamterene, chlorthalidone etc.; sympatholytics such as methyldopa, clonidine, guanabenz, reserpine; and other agents suitable for the treatment of high blood pressure, heart failure or vascular disorders associated with diabetes or renal disorders such as acute or chronic renal failure in humans and animals. Such combinations can be used separately or in products which comprise a plurality of components.

Further substances which can be used in combination with the compounds of the formulae (I) or (IA) are the compounds of classes (i) to (ix) on page 1 of WO 02/40007 (and the preferences and examples detailed further therein) and the substances mentioned on pages 20 and 21 of WO 03/027091.

The dosage may vary within wide limits and must of course be adapted to the individual circumstances in each individual case. In general, a daily dose appropriate for oral administration ought to be from about 3 mg to about 3 g, preferably about 10 mg to about 1 g, e.g. approximately 300 mg per adult person (70 kg), divided into preferably 1-3 single doses, which may be for example of equal size, although the stated upper limit may also be exceeded if this proves to be indicated, and children usually receive a reduced dose appropriate for their age and body weight.

### EXAMPLES

The following examples illustrate the present invention. All temperatures are stated in degrees Celsius and pressures in mbar. Unless mentioned otherwise, the reactions take place at room temperature. The abbreviation “Rf=xx (A)” means for example that the Rf is found in solvent system A to be xx. The ratio of amounts of solvents to one another is always stated in parts by volume. Chemical names for final products and intermediates have been generated on the basis of the chemical structural formulae with the aid of the AutoNom 2000 (Automatic Nomenclature) program. Unless mentioned otherwise, the absolute stereochemistry of all four asymmetric C atoms in the main chain is “S” in each case.

HPLC gradients on Hypersil BDS C-18 (5 um); column: 4×125 mm

I 90% water*/10% acetonitrile* to 0% water*/100% acetonitrile* in 5 minutes+2.5 minutes (1.5 ml/min)
II 95% water*/5% acetonitrile* to 0% water*/100% acetonitrile* in 40 minutes (0.8 ml/min) * contains 0.1% trifluoroacetic acid

The following abbreviations are used:
M.p. melting point (temperature)
Rf ratio of distance migrated by a substance to the distance of the solvent front from the starting point in thin-layer chromatography
Rt retention time of a substance in HPLC (in minutes)

### General Method A: (Azide Reduction)

A solution of 1 mmol of “azide derivative” in 10-20 ml of ethanol and ethanolamine (1 equiv) is hydrogenated in the presence of 200-400 mg of Pd/C 10% (moist) at 0° C. for 1-3 hours. The reaction mixture is clarified by filtration and the catalyst is washed with ethanol. The filtrate is evaporated. The title compound is obtained from the residue by flash chromatography (SiO₂ 60 F).

### General Method B: (Lactone Amidation I)

A mixture of 1 mmol of “lactone”, “amine” (10-30 equiv.) and 2-hydroxypyridine (1 equiv.) is stirred at 65° C. for 2-24 hours. The reaction mixture is cooled to room temperature, evaporated, mixed with 1M aqueous sodium bicarbonate solution and extracted with tert-butyl methyl ether (2×). The combined organic phases are washed with water and brine, dried with sodium sulphate and evaporated. The title compound is obtained from the residue by flash chromatography (SiO₂ 60 F).

### General Method C: (Lactone Amidation II)

A solution of 1.2 mmol of “amine” in 1-2 ml of toluene is added to a solution of 1.1 mmol of trimethylaluminium solution (2M in heptane) at −78° C. The reaction mixture is warmed to room temperature, stirred for a further 30-60 minutes and then evaporated. A solution of 1 mmol of “lactone” in 2 ml of toluene is added to the residue, and the mixture is stirred at 80° C. for 2-4 hours. The reaction mixture is cooled to room temperature and, after addition of 10 ml of 1N HCl, stirred for 30 minutes. The reaction mixture is diluted with brine and extracted with toluene (2×)—the combined organic phases are dried with sodium sulphate and evaporated. The title compound is obtained from the residue by flash chromatography (SiO₂ 60 F).

### General Method D: (Desilylation)

1.5 mmol of tetrabutylammonium fluoride solution (1M in tetrahydrofuran) are added to a solution of 1 mmol of “silyl ether” in 10-15 ml of tetrahydrofuran at 0° C. The reaction mixture is stirred at room temperature for 2-4 hours, poured into 1M aqueous sodium bicarbonate solution and extracted with tert-butyl methyl ether (2×). The combined organic phases are washed with brine, dried with sodium sulphate and evaporated. The title compound is obtained from the residue by flash chromatography (SiO₂ 60 F).

### General Method E: (Chloro Enamine Coupling)

1.2-1.8 mmol of (1-chloro-2-methylpropenyl)dimethylamine (chloro enamine) are added to a solution of 1 mmol of “acid” in 10 ml of dichloromethane at 0° C. After 2-4 hours, the reaction mixture is evaporated and the residue is dissolved in 6 ml of dichloromethane—this solution is added dropwise to the solution of 1.25 mmol of “amine” and 1.1 mmol of triethylamine in 6 ml of dichloromethane at 0° C. over the course of 2-10 minutes. The reaction mixture is stirred at room temperature for 1-2 hours, poured into water and extracted with tert-butyl methyl ether (2×). The combined organic phases are washed with brine, dried with sodium sulphate and evaporated. The title compound is obtained from the residue by flash chromatography (SiO₂ 60 F).

### General Method F: (Lactone Opening/Silylation)

A solution of 1 mmol of “lactone” in 5 ml of dioxane is mixed with 5 ml of water and 1.1 mmol of lithium hydroxide monohydrate. After 4-6 hours, the reaction mixture is mixed with ice and 1M aqueous citric acid solution and extracted with tert-butyl methyl ether (3×). The combined organic phases are washed with cold water and cold brine, dried with sodium sulphate and evaporated at room temperature. The residue is dissolved without delay in 8 ml of N,N-dimethylformamide and then 5 mmol of tert-butylchlorodimethylsilane and 8.8 mmol of imidazole are added. After 10-20 hours, the reaction mixture is evaporated—the residue is mixed with diethyl ether and water and adjusted to pH 4 with 1M aqueous citric acid solution and then the organic phase is separated off. The aqueous phase is extracted again with diethyl ether (3×)—the combined organic phases are washed with brine, dried with sodium sulphate and evaporated. The residue is dissolved in 3 ml of tetrahydrofuran, and 3 ml of water and 9 ml of acetic acid are successively added. After 3-4 hours, the reaction mixture is poured into ice-water and extracted with diethyl ether (2×)—the combined organic phases are washed with water and brine, dried with sodium sulphate and evaporated. The title compound is obtained from the residue by flash chromatography (SiO₂ 60 F).

### General Method G: (Grignard Reaction)

A solution of 1.33 mmol of “aryl bromide” in 2.70 ml of tetrahydrofuran is cooled to −78° C., and 2 mmol of N-methylmorpholine are added. Then 1.33 mmol of butyllithium solution (1.6M in hexane) are added at −78° C. The reaction mixture is stirred at −78° C. for 5 minutes, and 2 mmol of magnesium bromide solution (0.3M, freshly prepared from 2 mmol of Mg turnings and 2 mmol of 1,2-dibromoethane in 6.67 ml of tetrahydrofuran at 60° C.) are added. The reaction mixture is stirred at −78° C. and, after 5 minutes, a solution of 1 mmol of 2-[2-azido-2-(4-isopropyl-5-oxotetrahydrofuran-2-yl)ethyl]-3-methylbutyraldehyde [173154-02-4] in 1 ml of tetrahydrofuran is added at −78° C. The reaction mixture is then stirred at −78° C. for 15 minutes and quenched with 1M aqueous ammonium chloride solution. It is extracted with tert-butyl methyl ether (3×). The combined organic phases are washed with brine, dried with sodium sulphate and evaporated. The title compound is obtained from the residue by flash chromatography (SiO₂ 60 F).

### General Method H: (Alcohol Methoxyacetylation)

2.6 mmol of pyridine, 2.4 mmol of methoxyacetyl chloride and 0.1 mmol of 4-dimethylaminopyridine are successively added to a solution of 1 mmol of “alcohol” in 13.5 ml of toluene at 0° C. The ice bath is removed and the reaction mixture is stirred at room temperature for 2 hours. The reaction mixture is poured into 0.5M HCl and then the organic phase is separated off. The aqueous phase is extracted again with diethyl ether (3×)—the combined organic phases are washed with brine, dried with sodium sulphate and evaporated. The title compound is obtained from the residue by flash chromatography (SiO₂ 60 F).

### General Method I: (Azide and Methoxyacetoxy Reduction)

A solution of 1 mmol of “azide methoxyacetoxy derivative” in 25 ml of ethanol and ethanolamine (1 mmol) is hydrogenated in the presence of 600 mg of Pd/C 10% (dry) at room temperature for 2-5 hours. The reaction mixture is clarified by filtration and the catalyst is washed with ethanol. The filtrate is evaporated. The residue is treated with 1M sodium bicarbonate solution and extracted with tert-butyl methyl ether (3×)—the combined organic phases are dried with sodium sulphate and evaporated. The title compound is obtained from the residue by flash chromatography (SiO₂ 60 F).

General Method J: (Boc Protection of Amine) 2 mmol of N,N-diisopropylethylamine and 2 mmol of di-tert-butyl dicarbonate are successively added to a solution of 1 mmol of “amine” in 22 ml of dichloromethane at 0° C. The reaction mixture is warmed to room temperature and stirred at room temperature overnight. The reaction mixture is poured into water and then the organic phase is separated off. The aqueous phase is again extracted with dichloromethane (2×)—the combined organic phases are washed with brine, dried with sodium sulphate and evaporated. The title compound is obtained from the residue by flash chromatography (SiO₂ 60 F).

### General Method K: (Boc Deprotection of Amine)

50 mmol of trifluoroacetic acid are added to a solution of 1 mmol of “amine” in 20 ml of dichloromethane at 0° C. The reaction mixture is stirred at 0° C. for 1-3 hours. The reaction mixture is neutralized with 1M sodium bicarbonate solution, and the aqueous phase is extracted with tert-butyl methyl ether (3×)—the combined organic phases are washed with brine, dried with sodium sulphate and evaporated. The title compound is obtained from the residue by flash chromatography (SiO₂ 60 F).

### General Method L: (Phenol Alkylation)

A solution of 1 mmol of “phenol” in 2 ml of dimethylformamide is mixed with 1.5 mmol of potassium carbonate and 1.1 mmol of 1-chloro-3-methoxypropane. The reaction mixture is stirred at 100° C. for 11 hours. The reaction mixture is filtered and evaporated. The residue is partitioned between ethyl acetate and water/brine 9:1. The phases are separated, the aqueous phase is extracted with ethyl acetate (2×)—the combined organic phases are washed with brine, dried with sodium sulphate and evaporated. The title compound is obtained from the residue by flash chromatography (SiO₂ 60 F).

### Example 1

### 5-Amino-4-hydroxy-2-isopropyl-7-[4-methoxy-3-(3-methoxypropoxy)benzyl]-8-methylnonanoic acid prop-2-ynylamide

Firstly 1.5 ml of methanol and 0.35 ml of conc. ammonia 25% and then 0.228 g of triphenylphosphine are added to a mixture of 0.300 g of 5-azido-4-hydroxy-2-isopropyl-7-[4-methoxy-3-(3-methoxypropoxy)benzyl]-8-methylnonanoic acid prop-2-ynylamide in 1.6 ml of tetrahydrofuran and 0.38 ml of water at room temperature. After 65 hours, the reaction mixture is poured into water and extracted with tert-butyl methyl ether (3×)—the combined organic phases are washed successively with water and brine, dried with sodium sulphate and evaporated. The title compound is obtained as a colourless oil from the residue by flash chromatography (SiO₂ 60 F). Rf=0.19 (dichloromethane/methanol/conc. ammonia 25% 200:20:1); Rt=3.73 (gradient I).

The starting materials are prepared as follows:

### a) 5-Azido-4-hydroxy-2-isopropyl-7-[4-methoxy-3-(3-methoxypropoxy)benzyl]-8-methylnonanoic acid prop-2-ynylamide

0.42 g of 5-azido-4-(tert-butyldimethylsilanyloxy)-2-isopropyl-7-[4-methoxy-3-(3-methoxypropoxy)benzyl]-8-methylnonanoic acid prop-2-ynylamide is reacted in analogy to Method D. The title compound is obtained as a yellowish oil. Rf=0.24 (EtOAc/heptane 1:1); Rt=4.85 (gradient I).

### b) 5-Azido-4-(tert-butyldimethylsilanyloxy)-2-isopropyl-7-[4-methoxy-3-(3-methoxypropoxy)benzyl]-8-methylnonanoic acid prop-2-ynylamide

0.40 g of 5-azido-4-(tert-butyldimethylsilanyloxy)-2-isopropyl-7-[4-methoxy-3-(3-methoxypropoxy)benzyl]-8-methylnonanoic acid and 0.055 ml of prop-2-ynylamine are reacted in analogy to Method E. The crude title compound is employed in the next stage. Rt=6.44 (gradient I).

### c) 5-Azido-4-(tert-butyldimethylsilanyloxy)-2-isopropyl-7-[4-methoxy-3-(3-methoxypropoxy)benzyl]-8-methylnonanoic acid

0.933 g of 5-{1-azido-3-[4-methoxy-3-(3-methoxypropoxy)benzyl]-4-methylpentyl}-3-isopropyldihydrofuran-2-one [324763-46-4] is reacted in analogy to Method F. The title compound is obtained as a yellowish oil. Rf=0.40 (EtOAc/heptane 1:1); Rt=6.54 (gradient I).

### Example 2

### 5-Amino-4-hydroxy-2-isopropyl-7-[4-methoxy-3-(3-methoxypropoxy)benzyl]-8-methylnonanoic acid (1-methylpiperidin-4-yl)amide

0.499 g of 5-azido-4-hydroxy-2-isopropyl-7-[4-methoxy-3-(3-methoxypropoxy)benzyl]-8-methylnonanoic acid (1-methylpiperidin-4-yl)amide is reacted in analogy to Method A. The title compound is obtained as a yellowish oil. Rf=0.12 (dichloromethane/methanol/conc. ammonia 25% 80:10:1); Rt=3.18 (gradient I).

The starting material is prepared as follows:

### a) 5-Azido-4-hydroxy-2-isopropyl-7-[4-methoxy-3-(3-methoxypropoxy)benzyl]-8-methylnonanoic acid (1-methylpiperidin-4-yl)amide

0.466 g of 5-{1-azido-3-[4-methoxy-3-(3-methoxypropoxy)benzyl]-4-methylpentyl}-3-isopropyldihydrofuran-2-one [324763-46-4] and 1.165 g of 1-methylpiperidin-4-ylamine are reacted in analogy to Method B. The title compound is obtained as a colourless oil. Rt=4.13 (gradient I).

### Example 3

### 5-Amino-4-hydroxy-2-isopropyl-7-[4-methoxy-3-(3-methoxypropoxy)benzyl]-8-methylnonanoic acidpiperidin-4-ylamide dihydrochloride

0.712 g of benzyl 4-{5-azido-4-hydroxy-2-isopropyl-7-[4-methoxy-3-(3-methoxypropoxy)-benzyl]-8-methylnonanoylamino}piperidine-1-carboxylate is reacted in analogy to Method A. The crude product is dissolved in a little tert-butanol and, after addition of 2 drops of 4N HCl/dioxane, frozen in liquid nitrogen and lyophilized under high vacuum overnight. The title compound is obtained from the residue as a white solid. Rf=0.17 (dichloromethane/methanol/conc. ammonia 25% 40:10:1); Rt=3.11 (gradient I).

The starting material is prepared as follows:

### a) Benzyl 4-{5-azido-4-hydroxy-2-isopropyl-7-[4-methoxy-3-(3-methoxypropoxy)benzyl]-8-methylnonanoylamino}piperidine-1-carboxylate

0.466 g of 5-{1-azido-3-[4-methoxy-3-(3-methoxypropoxy)benzyl]-4-methylpentyl}-3-isopropyldihydrofuran-2-one [324763-46-4], 1.21 g of benzyl 4-aminopiperidine-1-carboxylate and 1.05 ml of triethylamine are reacted in analogy to Method B. The title compound is obtained as a yellowish oil. Rt=5.34 (gradient I).

### Example 4

### 5-Amino-4-hydroxy-2-isopropyl-7-[4-methoxy-3-(3-methoxypropoxy)benzyl]-8-methylnonanoic acidpyrrolidin-3(R)-ylamide dihydrochloride

0.607 g of 5-azido-4-hydroxy-2-isopropyl-7-[4-methoxy-3-(3-methoxypropoxy)benzyl]-8-methylnonanoic acidpyrrolidin-3(R)-ylamide is reacted in analogy to Method A. The chromatographed product is dissolved in 10 ml of dichloromethane, dried with sodium sulphate and filtered, and 1 ml of 4N HCl/dioxane is added and evaporated. The residue is dissolved in 2 ml of tert-butanol, frozen in liquid nitrogen and lyophilized under high vacuum overnight. The title compound is obtained as a white solid from the residue. Rf=0.08 (dichloromethane/methanol/conc. ammonia 25% 40:20:1); Rt=3.12 (gradient I).

The starting material is prepared as follows:

### a) 5-Azido-4-hydroxy-2-isopropyl-7-[4-methoxy-3-(3-methoxypropoxy)benzyl]-8-methylnonanoic acidpyrrolidin-3(R)-ylamide

A solution of 0.83 g of tert-butyl 3(R)-{5-azido-4-hydroxy-2-isopropyl-7-[4-methoxy-3-(3-methoxypropoxy)benzyl]-8-methylnonanoylamino}pyrrolidine-1-carboxylate in 12 ml of 4N HCl/dioxane is stirred at 0° C. for 1 hour and then evaporated. The title compound is obtained as a yellow oil from the residue by flash chromatography (SiO₂ 60 F). Rf=0.25 (dichloromethane/methanol/conc. ammonia 25% 40:10:1); Rt=4.03 (gradient I).

The following compounds are prepared in an analogous manner to the process described in Examples 1-4:

### Examples

5 5-Amino-4-hydroxy-2-isopropyl-7-[4-methoxy-3-(3-methoxypropoxy)benzyl]-8-methylnonanoic acid (tetrahydropyran-4-yl)amide

yellowish oil; Rf=0.37 (dichloromethane/methanol/conc. ammonia 25% 200:20:1); Rt=3.52 (gradient I).
6 5-Amino-4-hydroxy-2-isopropyl-7-[4-methoxy-3-(3-methoxypropoxy)benzyl]-8-methylnonanoic acid (1-methylpyrrolidin-3(S)-yl)amide dihydrochloride

white solid; Rf=0.21 (dichloromethane/methanol/conc. ammonia 25% 40:20:1); Rt=3.18 (gradient I).
7 5-Amino-4-hydroxy-2-isopropyl-7-[4-methoxy-3-(3-methoxypropoxy)benzyl]-8-methylnonanoic acid (1-methylpyrrolidin-3(R)-yl)amide dihydrochloride

white solid; Rf=0.17 (dichloromethane/methanol/conc. ammonia 25% 40:20:1); Rt=3.14 (gradient I).
8 5-Amino-4-hydroxy-2-isopropyl-7-[4-methoxy-3-(3-methoxypropoxy)benzyl]-8-methylnonanoic acid pyrrolidin-3(S)-ylamide dihydrochloride

white solid; Rf=0.09 (dichloromethane/methanol/conc. ammonia 25% 40:20:1); Rt=3.10 (gradient I).
9 5-Amino-4-hydroxy-2-isopropyl-7-[4-methoxy-3-(3-methoxypropoxy)benzyl]-8-methylnonanoic acid (1,1-dimethylprop-2-ynyl)amide
10 5-Amino-4-hydroxy-2-isopropyl-7-[4-methoxy-3-(3-methoxypropoxy)benzyl]-8-methylnonanoic acid but-2-ynylamide
11 5-Amino-4-hydroxy-2-isopropyl-7-[4-methoxy-3-(3-methoxypropoxy)benzyl]-8-methylnonanoic acid azetidin-3-ylamide dihydrochloride
12 5-Amino-4-hydroxy-2-isopropyl-7-[4-methoxy-3-(3-methoxypropoxy)benzyl]-8-methylnonanoic acid (1-methylazetidin-3-yl)amide dihydrochloride
13 5-Amino-4-hydroxy-2-isopropyl-7-[4-methoxy-3-(3-methoxypropoxy)benzyl]-8-methylnonanoic acid (azetidin-3-ylmethyl)amide

colourless glass; Rf=0.20 (dichloromethane/methanol/conc. ammonia 25% 40:10:1); Rt=3.14 (gradient I).
14 5-Amino-4-hydroxy-2-isopropyl-7-[4-methoxy-3-(3-methoxypropoxy)benzyl]-8-methylnonanoic acid (1-methylazetidin-3-ylmethyl)amide dihydrochloride
21 5-Amino-4-hydroxy-2-isopropyl-7-[3-(3-methoxypropoxy)-4-methylbenzyl]-8-methylnonanoic acid (tetrahydropyran-4-yl)amide

beige oil; Rf=0.18 (dichloromethane/methanol/conc. ammonia 25% 200:20:1); Rt=4.07 (gradient I).
22 5-Amino-4-hydroxy-2-isopropyl-7-[4-methoxy-3-(4-methoxybutyl)benzyl]-8-methylnonanoic acid (tetrahydropyran-4-yl)amide

yellow oil; Rf=0.20 (dichloromethane/methanol/conc. ammonia 25% 200:20:1); Rt=4.19 (gradient I).
23 5-Amino-4-hydroxy-2-isopropyl-7-[4-methoxy-3-(3-methoxypropoxy)benzyl]-8-methylnonanoic acid (1,2,2,6,6-pentamethyl piperidin-4-yl)amide dihydrochloride
24 5-Amino-4-hydroxy-2-isopropyl-7-[4-methoxy-3-(3-methoxypropoxy)benzyl]-8-methylnonanoic acid (2,2,6,6-tetramethylpiperidin-4-yl)amide

colourless oil; Rf=0.08 (dichloromethane/methanol/conc. ammonia 25% 200:20:1); Rt=3.33 (gradient I).
25 5-Amino-4-hydroxy-2-isopropyl-7-[4-methoxy-3-(3-methoxypropoxy)benzyl]-8-methylnonanoic acid (1-isopropylpiperidin-4-yl)amide

yellowish oil; Rf=0.39 (dichloromethane/methanol/conc. ammonia 25% 80:10:1); Rt=3.21 (gradient I).
26 5-Amino-4-hydroxy-2-isopropyl-7-[4-methoxy-3-(3-methoxypropoxy)benzyl]-8-methylnonanoic acid (1-isobutylpiperidin-4-yl)amide dihydrochloride
27 5-Amino-4-hydroxy-2-isopropyl-7-[4-methoxy-3-(3-methoxypropoxy)benzyl]-8-methylnonanoic acid (1-cyclopropylmethylpiperidin-4-yl)amide dihydrochloride
28 5-Amino-4-hydroxy-2-isopropyl-7-[4-methoxy-3-(3-methoxypropoxy)benzyl]-8-methylnonanoic acid (1-propylpiperidin-4-yl)amide

yellowish oil; Rf=0.50 (dichloromethane/methanol/conc. ammonia 25% 80:10:1); Rt=3.23 (gradient I).
29 5-Amino-4-hydroxy-2-isopropyl-7-[4-methoxy-3-(3-methoxypropoxy)benzyl]-8-methylnonanoic acid (1-ethylpiperidin-4-yl)amide

yellowish oil; Rf=0.47 (dichloromethane/methanol/conc. ammonia 25% 80:10:1); Rt=3.15 (gradient I).
36 5-Amino-4-hydroxy-2-isopropyl-7-[4-methoxy-3-(3-methoxypropoxy)benzyl]-8-methylnonanoic acid (tetrahydrofuran-2(S)-ylmethyl)amide

yellowish oil; Rf=0.36 (dichloromethane/methanol/conc. ammonia 25% 200:20:1); Rt=3.81 (gradient I).
39 5-Amino-4-hydroxy-2-isopropyl-7-[4-methoxy-3-(3-methoxypropoxy)benzyl]-8-methylnonanoic acid (furan-2-ylmethyl)amide

yellowish oil; Rf=0.19 (dichloromethane/methanol/conc. ammonia 25% 200:20:1); Rt=3.92 (gradient I).
44 5-Amino-4-hydroxy-2-isopropyl-7-[4-methoxy-3-(3-methoxypropoxy)benzyl]-8-methylnonanoic acid (tetrahydropyran-4-ylmethyl)amide

colourless oil; Rf=0.31 (dichloromethane/methanol/conc. ammonia 25% 200:20:1); Rt=3.61 (gradient I).
60 5-Amino-4-hydroxy-2-isopropyl-7-[4-methoxy-3-(3-methoxypropoxy)benzyl]-8-methylnonanoic acid (furan-3-ylmethyl)amide
63 5-Amino-4-hydroxy-2-isopropyl-7-[4-methoxy-3-(3-methoxypropoxy)benzyl]-8-methylnonanoic acid ((cis)-1-oxaspiro[2.5]oct-6-yl)amide

The starting materials are prepared as follows:

### a) (cis)-1-Oxaspiro[2.5]oct-6-ylamine and (trans)-1-oxaspiro[2.5]oct-6-ylamine

A solution of 3.55 mmol of benzyl (1-oxaspiro[2.5]oct-6-yl)carbamate [142010-03-5] in 80 ml of methanol is hydrogenated in the presence of 0.13 mmol of Pd/C 10% (moist) at 0° C. for 1 hour. The reaction mixture is clarified by filtration and the catalyst is washed with ethanol. The filtrate is evaporated. The title compounds are identified from the residue by flash chromatography (SiO₂ 60 F) on the basis of their Rf.
64 5-Amino-4-hydroxy-2-isopropyl-7-[4-methoxy-3-(3-methoxypropoxy)benzyl]-8-methylnonanoic acid ((trans)-1-oxaspiro[2.5]oct-6-yl)amide

The starting material is prepared as described in Example 63.
65 5-Amino-4-hydroxy-2-isopropyl-7-[4-methoxy-3-(3-methoxypropoxy)benzyl]-8-methylnonanoic acid (tetrahydropyran-2(R)-ylmethyl)amide

The starting materials are prepared as follows:

### a) C-(Tetrahydropyran-2(R)-yl)methylamine

A solution of 2.62 mmol of 2(R)-azidomethyltetrahydropyran in 150 ml of methanol is hydrogenated in the presence of 0.03 mmol of Pd/C 10% (moist) until conversion is complete. The reaction mixture is clarified by filtration and the catalyst is washed with ethanol. The filtrate is evaporated. The crude title compounds are identified from the residue on the basis of their Rf.

### b) 2(R)-Azidomethyltetrahydropyran

A solution of 5 mmol of tetrahydropyran-2(R)-ylmethyl methanesulphonate and 55 mmol of sodium azide in 50 ml of dimethyl sulphoxide is stirred at room temperature for 20 hours. It is then diluted with water and tert-butyl methyl ether and washed with brine. The aqueous phase is extracted with tert-butyl methyl ether (2×). The combined organic phases are dried with sodium sulphate and evaporated. The crude title compound is identified from the residue on the basis of its Rf.

### c) Tetrahydropyran-2(R)-ylmethyl methanesulphonate

50 mmol of triethylamine and 20 mmol of methanesulphonyl chloride are successively added to a solution of 10 mmol of (tetrahydropyran-2(R)-yl)methanol [70766-06-2] in 100 ml of dichloromethane at 0° C. The mixture is stirred at 0° C. for 1 hour, diluted with dichloromethane and washed with 1M HCl. The organic phase is dried with sodium sulphate and evaporated. The crude title compound is identified from the residue on the basis of its Rf.
66 5-Amino-4-hydroxy-2-isopropyl-7-[4-methoxy-3-(3-methoxypropoxy)benzyl]-8-methylnonanoic acid (tetrahydropyran-2(S)-ylmethyl)amide

The starting material is prepared in analogy to Example 65 a-c from (tetrahydropyran-2(S)-yl)methanol [51450-44-3].
67 5-Amino-4-hydroxy-2-isopropyl-7-[4-methoxy-3-(3-methoxypropoxy)benzyl]-8-methylnonanoic acid (tetrahydropyran-3(R)-yl)amide

The starting material is prepared in analogy to Example 65 a-c from tetrahydropyran-3(S)-ol [72886-97-6].
68 5-Amino-4-hydroxy-2-isopropyl-7-[4-methoxy-3-(3-methoxypropoxy)benzyl]-8-methylnonanoic acid (tetrahydropyran-3(S)-yl)amide

The starting material is prepared in analogy to Example 65 a-c from tetrahydropyran-3(R)-ol [100937-76-6].
75 5-Amino-4-hydroxy-2-isopropyl-7-[4-methoxy-3-(3-methoxypropoxy)benzyl]-8-methylnonanoic acid ([1,4]dioxan-2(R,S)-ylmethyl)amide

colourless wax; Rf=0.26 (dichloromethane/methanol/conc. ammonia 25% 200:20:1); Rt=3.56 (gradient I).
76 5-Amino-4-hydroxy-2-isopropyl-7-[4-methoxy-3-(3-methoxypropoxy)benzyl]-8-methylnonanoic acid[1,4]dioxepan-6-ylamide

colourless wax; Rf=0.18 (dichloromethane/methanol/conc. ammonia 25% 200:20:1); Rt=3.56 (gradient I).

The starting material is prepared as follows:

### a) [1,4]-Dioxepan-6-ylamine

A solution of 0.095 g of 6-azido[1,4]dioxepane in 2 ml of ethyl acetate is mixed with 0.053 g of 10% palladium/C (50%, moist with water), and the reaction mixture is hydrogenated with the aid of a balloon at room temperature for 45 minutes. The catalyst is filtered off through Hyflo. The filtrate is then employed directly in the amide coupling.

### b) 6-Azido[1,4]dioxepane

A solution of 0.400 g of [1,4]dioxepan-6-yl toluene-4-sulphonate in 5 ml of N,N-dimethylformamide is mixed with 0.185 g of sodium azide. The reaction mixture is heated at 60° C. for 20 hours and then poured into water. The mixture is extracted with tert-butyl methyl ether, and the combined organic extracts are washed with brine, dried with sodium sulphate and evaporated. The title compound is obtained as a colourless oil from the residue by flash chromatography (SiO₂ 60 F). Rf=0.42 (EtOAc/heptane 1:1).

### c) [1,4]Dioxepan-6-yl toluene-4-sulphonate

A solution of 0.470 g of [1,4]dioxepan-6-ol [28544-95-8] in 10 ml of dichloromethane is mixed with 0.836 ml of triethylamine and 0.050 g of N,N-dimethylaminopyridine. At room temperature, 0.824 g of toluene-4-sulphonyl chloride is added in portions and the reaction mixture is then stirred at room temperature for 3 hours. The reaction mixture is poured into saturated aqueous sodium bicarbonate solution. The mixture is extracted with tert-butyl methyl ether, and the combined extracts are washed with brine, dried with sodium sulphate and evaporated. The title compound is obtained as a colourless oil from the residue by flash chromatography (SiO₂ 60 F). Rf=0.29 (EtOAc/heptane 1:1); Rt=3.80 (gradient I).
77 5-Amino-4-hydroxy-2-isopropyl-7-[4-methoxy-3-(3-methoxypropoxy)benzyl]-8-methylnonanoic acid ((exo)-3-oxabicyclo[3.1.0]hex-6(R,S)-ylmethyl)amide

yellow oil; Rf=0.21 (dichloromethane/methanol/conc. ammonia 25% 200:20:1); Rt=3.61 (gradient I).

The starting material is prepared as follows:

### a) (exo)-C-[1-(3-Oxabicyclo[3.1.0]hex-6-yl)]methylamine

65.3 g of lithium aluminium hydride (pellets) are added in portions to a stirred mixture of 70 g of (exo)-3-oxabicyclo[3.1.0]hexane-6-carboxamide and 1.1 l of tetrahydrofuran at 0° C. The reaction mixture is stirred at room temperature for 19 hours. The resulting mixture is cooled to 0° C. and 100 ml of water, 100 ml of 1M NaOH and 300 ml of water are successively added dropwise. The resulting suspension is clarified by filtration through Hyflo, and the filtrate is evaporated. The title compound is obtained as a colourless liquid from the residue by distillation. b.p. 65-75° C. under 15 mbar.

### b) (exo)-3-Oxabicyclo[3.1.0]hexane-6-carboxamide

A mixture of 95.0 g of ethyl (exo)-3-oxabicyclo[3.1.0]hexane-6-carboxylate [CAS 81056-11-3] and 500 ml of aqueous 30% strength ammonia solution is stirred at room temperature for 3 days. The resulting emulsion is evaporated to dryness and the residue is dried in vacuo. The crude title compound is obtained as a brown solid.
78 5-Amino-4-hydroxy-2-isopropyl-7-[4-methoxy-3-(3-methoxypropoxy)benzyl]-8-methylnonanoic acid ((exo,endo)-2-oxabicyclo[3.1.0]hex-6-ylmethyl)amide

The starting material is prepared in analogy to Example 77 a from (exo,endo)-2-oxabicyclo[3.1.0]hexane-6-carboxamide [89598-52-7].
79 5-Amino-4-hydroxy-2-isopropyl-7-[4-methoxy-3-(3-methoxypropoxy)benzyl]-8-methylnonanoic acid ((exo,endo)-3-oxabicyclo[3.1.0]hex-6-yl)amide

colourless oil; Rf=0.21 (dichloromethane/methanol/conc. ammonia 25% 200:20:1); Rt=3.50 (gradient I).

The starting material is prepared as follows:

### a) (exo)-(3-Oxabicyclo[3.1.0]hex-6-yl)amine

A mixture of 7.0 g of tert-butyl (exo)-(3-oxabicyclo[3.1.0]hex-6-yl)carbamate and 52 ml of 4M HCl (in dioxane) is stirred at 0° C. for 1 hour and then at room temperature for 2 hours. The resulting suspension is cooled in an ice bath and, after addition of 200 ml of tert-butyl methyl ether, the solid ((exo)-(3-oxabicyclo[3.1.0]hex-6-yl)amine hydrochloride) is filtered off with suction.

The hydrochloride is added to a stirred mixture of 40 ml of 50% strength sodium hydroxide solution and 75 ml of tert-butyl methyl ether. The organic phase is separated off, dried with sodium hydroxide (solid), evaporated and distilled at 100 mbar/40° C. The title compound is obtained as a pale yellowish liquid.

### b) tert-Butyl (exo)-(3-oxabicyclo[3.1.0]hex-6-yl)carbamate

A solution of 50.2 g of (exo)-3-oxabicyclo[3.1.0]hexane-6-carboxylic acid [CAS55780-88-6] and 450 ml of tert-butanol is warmed to 30° C. Then, over the course of 30 minutes, 58.7 ml of triethylamine and 91.4 ml of diphenylphosphoryl azide are added dropwise in parallel. The reaction mixture is stirred at 70° C. for 18 hours. The resulting mixture is evaporated, and the residue is mixed with 1 l of 1M HCl and extracted with ethyl acetate (3×). The organic phases are washed with water (1×),1M sodium bicarbonate solution and brine (1×), dried with sodium sulphate and filtered, and the filtrate is evaporated. The title compound is obtained in the form of white crystals from the residue by flash chromatography and crystallization. Rf=0.33 (EtOAc/heptane 1:1); b.p. 86-87° C.
80 5-Amino-4-hydroxy-2-isopropyl-7-[4-methoxy-3-(3-methoxypropoxy)benzyl]-8-methylnonanoic acid ((exo,endo)-2-oxabicyclo[3.1.0]hex-6-yl)amide

The starting material is prepared in analogy to Example 79 a-b from (exo,endo)-2-oxabicyclo[3.1.0]hexane-6-carboxylic acid [99418-15-2].
81 5-Amino-4-hydroxy-2-isopropyl-7-[4-methoxy-3-(3-methoxypropoxy)benzyl]-8-methylnonanoic acid (6-oxaspiro[2.5]oct-1(R)-yl)amide

colourless oil; Rf=0.21 (dichloromethane/methanol/conc. ammonia 25% 200:20:1); Rt=3.66 (gradient I).

The starting materials are prepared as follows:

### a) (R)-(6-Oxaspiro[2.5]oct-1-yl)amine

A solution of 0.528 g of (R)-6-oxaspiro[2.5]octane-1-carboxylic acid in 5 ml of tetrahydrofuran is mixed with 1.20 ml of triethylamine, and the solution is then cooled to 0° C. The solution is mixed with 0.657 ml of ethyl chloroformate, and the reaction mixture is stirred at 0° C. for 1 hour. A solution of 4.44 g of sodium azide in 5 ml of water is added and the reaction mixture is stirred at 0° C. for a further hour. The reaction mixture is diluted with water and extracted with tert-butyl methyl ether. The combined extracts are washed with brine, dried with sodium sulphate and evaporated. The residue is taken up in 5 ml of benzene, and the solution is heated to reflux for 2 hours and then evaporated. The residue is dissolved in 5 ml of tetrahydrofuran, and 5 ml of water and 0.347 g of lithium hydroxide are added. The reaction mixture is stirred at room temperature for 3 hours and then adjusted to pH 2 with 4M HCl. The tetrahydrofuran is distilled off, and the aqueous residue is adjusted to pH 12 with 2M NaOH. The aqueous phase is extracted with dichloromethane, and the combined organic extracts are dried over sodium sulphate and evaporated. The title compound is obtained as a yellow oil which is employed without further purification in the next stage. Rf=0.26 (dichloromethane/methanol/conc. ammonia 25% 200:20:1).

### b) (R)-6-Oxaspiro[2.5]octane-1-carboxylic acid

A solution of 3.700 g of (R)-4-benzyl-3-((R)-6-oxaspiro[2.5]octane-1-carbonyl)oxazolidin-2-one in 20 ml of tetrahydrofuran/water 3:1 is cooled to 0° C. 0.608 g of lithium hydroxide and 1.95 ml of hydrogen peroxide (30% in water) are added to the solution, which is stirred at room temperature for 6 hours. Saturated aqueous sodium thiosulphate solution is added to the reaction mixture, which is then extracted with tert-butyl methyl ether. The aqueous phase is adjusted to pH 2 with 4M HCl and extracted with dichloromethane. The combined extracts are washed with brine, dried with sodium sulphate and evaporated. The title compound is obtained as a colourless liquid and is employed without further purification for the next stage. Rf=0.55 (dichloromethane/methanol/water/acetic acid 150:54:10:1); Rt=2.10 (gradient I).

### c) (R)-4-Benzyl-3-((R)-6-oxaspiro[2.5]octane-1-carbonyl)oxazolidin-2-one and (R)-4-benzyl-3-((S)-6-oxaspiro[2.5]octane-1-carbonyl)oxazolidin-2-one

A solution of 118.81 g of (R)-4-benzyloxazolidin-2-one [102029-44-7] in 90 ml of anhydrous tetrahydrofuran is cooled to −75° C. under argon. A solution of n-butyllithium (1.6M in hexane) is added at −75°-−60° C. over 2 hours. The reaction mixture is stirred at −75° C. for 10 minutes and a solution of 110.37 g of 6-oxaspiro[2.5]octane-1-carbonyl chloride in 100 ml of tetrahydrofuran is added dropwise at −75°-−60° C. The reaction mixture is then slowly warmed to 20° C. and saturated aqueous ammonium chloride solution is added. The mixture is extracted with tert-butyl methyl ether, and the combined extracts are washed with brine, dried with sodium sulphate and evaporated. The title compounds are obtained as white solids from the residue by column chromatography (SiO₂ 60 F). Rf (diastereomer 1)=0.25 (EtOAc/heptane 1:2); Rt (diastereomer 1)=4.20 (gradient I); Rf (diastereomer 2)=0.21 (EtOAc/heptane 1:2); Rt (diastereomer 2)=4.27 (gradient I).

### d) 6-Oxaspiro[2.5]octane-1-carbonyl chloride

60.0 ml of oxalyl chloride are added to a solution of 98.73 g of 6-oxaspiro[2.5]octane-1-carboxylic acid in 500 ml of dichloromethane at 0° C. One drop of N,N-dimethylformamide is added, and the reaction solution is stirred at room temperature for 1 hour. The reaction solution is then evaporated and the crude product is employed directly for the next stage.

### e) 6-Oxaspiro[2.5]octane-1-carboxylic acid

A solution of 120.50 g of ethyl 6-oxaspiro[2.5]octane-1-carboxylate in 700 ml of ethanol/water 3.5:1 is mixed with 55.20 g of potassium hydroxide. The reaction mixture is heated at 60° C. for 4 hours, the ethanol is distilled off and the aqueous residue is diluted with water and extracted with tert-butyl methyl ether. The aqueous phase is adjusted to pH 2 with 4M hydrochloric acid and extracted with tert-butyl methyl ether. The combined organic extracts are washed with brine, dried with sodium sulphate and evaporated. The title compound is obtained as a yellowish oil and is employed without further purification for the next stage. Rt=2.13 (gradient I).

### f) Ethyl 6-oxaspiro[2.5]octane-1-carboxylate

51.93 g of sodium hydride (60% dispersion in oil) is taken up in 1100 ml of dry dimethyl sulphoxide under argon. 272 g of trimethylsulphoxonium iodide are added in portions over 20 minutes at room temperature, and the reaction mixture is then stirred at room temperature for 1.5 hours. 170.00 g of ethyl (tetrahydropyran-4-ylidene)acetate [130312-00-4] are added dropwise, and the reaction mixture is stirred at room temperature for 18 hours. The mixture is poured into ice and extracted with tert-butyl methyl ether. The combined organic extracts are washed with brine, dried with sodium sulphate and evaporated. The title compound is obtained as a colourless oil from the residue by vacuum distillation (68-70° C., 0.09 mbar). Rt=3.49 (gradient I).
82 5-Amino-4-hydroxy-2-isopropyl-7-[4-methoxy-3-(3-methoxypropoxy)benzyl]-8-methylnonanoic acid (6-oxaspiro[2.5]oct-1(S)-yl)amide

colourless oil; Rf=0.21 (dichloromethane/methanol/conc. ammonia 25% 200:20:1); Rt=3.69 (gradient I).

The starting materials are prepared as follows:

### a) (S)-(6-Oxaspiro[2.5]oct-1-yl)amine

The title compound is prepared in analogy to Example 81 a-b from (R)-4-benzyl-3-((S)-6-oxaspiro[2.5]octane-1-carbonyl)oxazolidin-2-one (Example 81c).
83 5-Amino-4-hydroxy-2-isopropyl-7-[4-methoxy-3-(3-methoxypropoxy)benzyl]-8-methylnonanoic acid (6-oxaspiro[2.5]oct-1(R)-ylmethyl)amide

yellowish oil; Rf=0.28 (dichloromethane/methanol/conc. ammonia 25% 200:20:1); Rt=3.74 (gradient I).

The starting material is prepared as follows:

### a) C—[(S)-1-(6-Oxaspiro[2.5]oct-1-yl)]methylamine

10.60 g of lithium aluminium hydride are taken up in 160 ml of dry tetrahydrofuran under argon. The suspension is cooled to 0° C., and a solution of 10.75 g of (S)-6-oxaspiro[2.5]octane-1-carboxamide in 60 ml of tetrahydrofuran is added dropwise. The reaction mixture is stirred at 0° C. for 4 hours and then 7.80 ml of water, followed by 34 ml of 3M NaOH and 30 ml of water, are added. The solid is filtered off through Hyflo, and the filtrate is washed with brine, dried with sodium sulphate and evaporated. The title compound is obtained as a yellowish oil and employed without further purification for the next stage.

### b) (S)-6-Oxaspiro[2,5]octane-1-carboxamide

A solution of 17.66 g of (S)-6-oxaspiro[2.5]octane-1-carboxylic acid (prepared in analogy to Example 81 b from (R)-4-benzyl-3-((S)-6-oxaspiro[2.5]octane-1-carbonyl)oxazolidin-2-one (Example 81c)) in 330 ml of ethyl acetate is mixed with 18.85 g of carbonyldiimidazole. The reaction solution is left to stand at room temperature for 6 hours and then 330 ml of 25% ammonium hydroxide solution are added. The reaction mixture is stirred at room temperature for 16 hours, the phases are separated, and the aqueous phase is extracted with ethyl acetate. The combined organic phases are washed with water until neutral, dried with sodium sulphate and evaporated. The title compounds are obtained from the residue by flash chromatography (SiO₂ 60 F) as a yellowish oil which may crystallize if sufficiently pure. Rf=0.28 (dichloromethane/methanol/conc. ammonia 25% 200:20:1).
84 5-Amino-4-hydroxy-2-isopropyl-7-[4-methoxy-3-(3-methoxypropoxy)benzyl]-8-methylnonanoic acid (6-oxaspiro[2.5]oct-1(R)-ylmethyl)amide

yellowish wax; Rf=0.25 (dichloromethane/methanol/conc. ammonia 25% 200:20:1); Rt=3.79 (gradient I).

The starting material is obtained in analogy to Example 83 from (R)-6-oxaspiro[2.5]octane-1-carboxylic acid (Example 81b).
85 5-Amino-4-hydroxy-2-isopropyl-7-[4-methoxy-3-(3-methoxypropoxy)benzyl]-8-methylnonanoic acid ((Z)-2-oxabicyclo[3.1.0]hex-1-ylmethyl)amide

The starting material is prepared as follows:

### a) C-((Z)-2-Oxabicyclo[3.1.0]hex-1-yl)methylamine

The title compound is prepared in analogy to Example 65 a-c from ((Z)-2-oxabicyclo[3.1.0]hex-1-yl)methanol.

### b) ((Z)-2-Oxabicyclo[3.1.0]hex-1-yl)methanol

2 mmol of a solution of borane-tetrahydrofuran complex (1M in tetrahydrofuran) are added to a solution of 1.0 mmol of (Z)-2-oxabicyclo[3.1.0]hexane-1-carbaldehyde in 10 ml of tetrahydrofuran at room temperature. The reaction mixture is stirred at room temperature for 2 hours, mixed with 10 ml of methanol and concentrated. The title compound is identified on the basis of its Rf from the residue by means of flash chromatography (SiO₂ 60 F).

### c) (Z)-2-Oxabicyclo[3.1.0]hexane-1-carbaldehyde

A solution of 0.66 mmol of 1(Z)-propenyl-(Z)-2-oxabicyclo[3.1.0]hexane [164118-97-2] and 0.80 mmol of N-methylmorpholine N-oxide hydrate in 20 ml of tetrahydrofuran/water/tert-butanol 4:4:1 is stirred at room temperature for 30 minutes. A 4% strength solution of osmium tetroxide (4 mol % in water) is added, and the reaction mixture is stirred at room temperature for 16 hours and then quenched with 2M sodium thiosulphate solution and ethyl acetate. The mixture is stirred at room temperature for 3 hours, the phases are separated, and the organic phase is washed with brine and evaporated. The residue is dissolved in 20 ml of ethyl acetate, and 1.0 mmol of lead tetraacetate is added. The suspension is stirred at room temperature for 10 minutes and filtered through a little SiO₂ 60 F and evaporated. The title compound is identified on the basis of its Rf from the residue by flash chromatography (SiO₂ 60 F).
87 5-Amino-4-hydroxy-2-isopropyl-7-[4-methoxy-3-(3-methoxypropoxy)benzyl]-8-methylnonanoic acid ((exo,endo)-2-oxabicyclo[4.1.0]hept-7-ylmethyl)amide

The starting material is prepared as follows:

### a) C-((exo,endo)-2-Oxabicyclo[4.1.0]hept-7-yl)methylamine

The title compound is prepared in analogy to Example 77 a-b from ethyl (exo,endo)-2-oxabicyclo[4.1.0]heptane-7-carboxylate [51197-04-7], [51144-35-5].
88 5-Amino-4-hydroxy-2-isopropyl-7-[4-methoxy-3-(3-methoxypropoxy)benzyl]-8-methylnonanoic acid (2-oxabicyclo[4.1.0]hept-7-yl)amide

The starting materials are prepared as follows:

### a) (exo,endo)-2-Oxabicyclo[4.1.0]hept-7-ylamine

The title compound is prepared in analogy to Example 79 a-b from (exo,endo)-2-oxabicyclo[4.1.0]heptane-7-carboxylic acid.

### b) (exo,endo)-2-Oxabicyclo[4.1.0]heptane-7-carboxylic acid

60 mmol of sodium periodate and 2 mmol of ruthenium(III) chloride hydrate are added to a two-phase solution of 1.5 mmol of ((exo,endo)-2-oxabicyclo[4.1.0]hept-7-yl)methanol [51197-04-7], [51144-35-5] in 30 ml of water, 20 ml of tetrachloromethane and 20 ml of acetonitrile. The reaction mixture is stirred at room temperature for 3 hours and then quenched with isopropanol. The reaction mixture is filtered through Hyflo and evaporated. The title compound is identified on the basis of its Rf from the residue by flash chromatography (SiO₂ 60 F).
89 5-Amino-4-hydroxy-2-isopropyl-7-[4-methoxy-3-(3-methoxypropoxy)benzyl]-8-methylnonanoic acid ((7,9trans,7exo)-7-methoxy-3-oxabicyclo[3.3.1]non-9-yl)amide

The starting materials are prepared as follows:

### a) (7,9trans,7exo)-7-Methoxy-3-oxabicyclo[3.3.1]non-9-ylamine

A solution of 0.9 mmol of (7exo)-7-methoxy-3-oxabicyclo[3.3.1]nonan-9-one in 5 ml of ethanol is mixed with a solution of 1.8 mmol of hydroxylamine hydrochloride in 0.5 ml of water and heated to reflux overnight. The reaction mixture is concentrated and partitioned between saturated sodium carbonate solution and diethyl ether. The phases are separated and the aqueous phase is extracted with diethyl ether (2×). The combined organic phases are dried with sodium sulphate and evaporated. The residue is dissolved in 5 ml of ethanol, and 12.8 mmol of zinc dust and 0.8 ml of glacial acetic acid are each added alternately in small portions over the course of 2 hours. The internal temperature must not exceed 50° C. during the addition. The reaction mixture is stirred at room temperature for 12 hours and filtered through Hyflo, and the filter cake is washed with cold ethanol. The solution is evaporated and the residue is partitioned between 4M NaOH and diethyl ether. The phases are separated and the aqueous phase is extracted with diethyl ether (2×). The combined organic phases are dried with sodium sulphate and evaporated. The title compounds are identified on the basis of their Rf from the residue by flash chromatography (SiO₂ 60 F).

### b) (7exo)-7-Methoxy-3-oxabicyclo[3.3.1]nonan-9-one

A solution of 1 mmol of (7exo)-7,9,9-trimethoxy-3-oxabicyclo[3.3.1]nonane in 10 ml of methanol is mixed with 10 ml of 2M HCl and heated to reflux for 3 hours. It is cooled to room temperature, and the reaction solution is concentrated. The residue is extracted with chloroform (3×). The combined organic phases are washed with sodium bicarbonate solution, dried with sodium sulphate and evaporated. The title compounds are identified on the basis of their Rf from the residue by flash chromatography (SiO₂ 60 F).

### c) (7exo)-7,9,9-Trimethoxy-3-oxabicyclo[3.3.1]nonane

A solution of 1.3 mmol of (7exo)-9,9-dimethoxy-3-oxabicyclo[3.3.1]nonan-7-ol in 10 ml of dimethylformamide is mixed with 5.2 mmol of methyl iodide and cooled to 0° C. 1.9 mmol of sodium hydride (60% dispersion in oil) are added in portions, and the mixture is stirred at 0° C. for 1 hour. Saturated sodium bicarbonate solution is added, and the mixture is extracted with tert-butyl methyl ether (2×). The combined organic phases are washed with water and brine, dried with sodium sulphate and evaporated. The title compounds are identified on the basis of their Rf from the residue by flash chromatography (SiO₂ 60 F).

### d) (7exo)-9,9-Dimethoxy-3-oxabicyclo[3.3.1]nonan-7-ol

A solution of 4.5 mmol of (7endo)-9,9-dimethoxy-3-oxabicyclo[3.3.1]nonan-7-ol in 100 ml of tetrahydrofuran is mixed with 5.4 mmol of benzoic acid and 5.4 mmol of triphenylphosphine. Then 4.5 mmol of diisopropyl azodicarboxylate are added dropwise, and the mixture is stirred at room temperature for 5 hours. Saturated sodium bicarbonate solution is added, the phases are separated, and the aqueous phase is extracted with dichloromethane (2×). The combined organic phases are dried with sodium sulphate and evaporated. The residue is dissolved in 35 ml of methanol and, after addition of 17 mmol of potassium carbonate, stirred at room temperature for 5 hours. The reaction mixture is evaporated and the residue is taken up in dichloromethane. Saturated ammonium chloride solution is added, the phases are separated, and the aqueous phase is extracted with dichloromethane (2×). The combined organic phases are dried with sodium sulphate and evaporated. The title compounds are identified on the basis of their Rf from the residue by flash chromatography (SiO₂ 60 F).

### e) (7endo)-9,9-Dimethoxy-3-oxabicyclo[3.3.1]nonan-7-ol

A solution of 1.89 mmol of 9,9-dimethoxy-3-oxabicyclo[3.3.1]nonan-7-one in 2 ml of tetrahydrofuran is added dropwise to a suspension of 3.68 mmol of lithium aluminium hydride in 4.5 ml of tetrahydrofuran at −20° C. The reaction mixture is stirred at −20° C. for 40 hours. Then, 144 μl of water, 144 μl of 5M NaOH and 432 μl of water are cautiously added successively and the mixture is stirred at room temperature for 25 minutes. The reaction mixture is filtered through Hyflo and evaporated. The title compound is identified on the basis of its Rf from the residue by flash chromatography (SiO₂ 60 F).

### f) 9,9-Dimethoxy-3-oxabicyclo[3.3.1]nonan-7-one

6 mmol of (7endo)-(9,9-dimethoxy-3-oxabicyclo[3.3.1]non-7-yl)phenylmethanone are added to a mixture of 6.7 mmol of potassium tert-butoxide in 25 ml of tert-butanol and 100 ml of hexamethylphosphoric triamide under an oxygen atmosphere. The reaction mixture is stirred at room temperature for 30 minutes and poured into ice-water. It is extracted with benzene/tetrahydrofuran (3×). The combined organic phases are washed with brine, dried with sodium sulphate and evaporated. The title compounds are identified by means of their Rf from the residue by flash chromatography (SiO₂ 60 F).

### g) (7endo)-(9,9-Dimethoxy-3-oxabicyclo[3.3.1]non-7-yl)phenylmethanone

A solution of 1 mmol of (7endo)-7-benzoyl-3-oxabicyclo[3.3.1]nonan-9-one in 20 ml of methanol is mixed with 1 ml of conc. H₂SO₄ and refluxed in a Dean-Stark apparatus for 3 hours. The reaction solution is cooled to room temperature and concentrated. The residue is extracted with chloroform (3×). The combined organic phases are washed with sodium bicarbonate solution, dried with sodium sulphate and evaporated. The title compounds are identified by means of their Rf from the residue by flash chromatography (SiO₂ 60 F).

### h) (7endo)-7-Benzoyl-3-oxabicyclo[3.3.1]nonan-9-one

140 mg of tosyl chloride are added to a solution of 50 mmol of tetrahydropyran-4-one [29943-42-8] and 11 ml of pyrrolidine in 50 ml of benzene, and the reaction mixture is refluxed in a Dean-Stark apparatus for 3 hours. The reaction solution is cooled to room temperature and evaporated. The residue is dissolved in 200 ml of acetonitrile and 60 mmol of triethylamine and, after addition of a solution of 50 mmol of 2-benzoyl-1,3-dichloropropane [39192-57-9] in 100 ml of acetonitrile, stirred at room temperature for 2 hours. The reaction mixture is quenched with water, stirred for 1 hour and evaporated. The residue is mixed with water and extracted with chloroform (2×). The combined organic phases are washed with 2N HCl and 1M sodium bicarbonate solution, dried with sodium sulphate and evaporated. The title compounds are identified by means of their Rf from the residue by flash chromatography (SiO₂ 60 F).
90 5-Amino-4-hydroxy-2-isopropyl-7-[4-methoxy-3-(3-methoxypropoxy)benzyl]-8-methylnonanoic acid ((7,9cis,7endo)-7-methoxy-3-oxabicyclo[3.3.1]non-9-yl)amide

The starting material is prepared as follows:

### a) (7,9cis,7endo)-7-Methoxy-3-oxabicyclo[3.3.1]non-9-ylamine

The title compound is prepared in analogy to Example 91 a-c from (7endo)-9,9-dimethoxy-3-oxabicyclo[3.3.1]nonan-7-ol (Example 91e).
96 5-Amino-4-hydroxy-2-isopropyl-7-[4-methoxy-3-(3-methoxypropoxy)benzyl]-8-methylnonanoic acid (tetrahydrofuran-3S-ylmethyl)amide

The starting material is prepared as follows:

### a) C-(Tetrahydrofuran-3-yl)methylamine

The title compound is prepared in analogy to Example 65 a-c from (tetrahydrofuran-3S-yl)-methanol [124391-75-9].
97 5-Amino-4-hydroxy-2-isopropyl-7-[4-methoxy-3-(3-methoxypropoxy)benzyl]-8-methylnonanoic acid (tetrahydrofuran-3R-ylmethyl)amide

The starting material is prepared as follows:

### a) C-(Tetrahydrofuran-3R-yl)methylamine

The title compound is prepared in analogy to Example 65 a-c from (tetrahydrofuran-3-yl)-methanol [124506-31-6].
98 5-Amino-4-hydroxy-2-isopropyl-7-[4-methoxy-3-(3-methoxypropoxy)benzyl]-8-methylnonanoic acid (tetrahydropyran-3R-ylmethyl)amide

The starting material is prepared as follows:

### a) C-(Tetrahydropyran-3R-yl)methylamine

The title compound is prepared in analogy to Example 89 a from tetrahydropyran-3S-carbaldehyde [141822-85-7].
100 5-Amino-4-hydroxy-2-isopropyl-7-[4-methoxy-3-(3-methoxypropoxy)benzyl]-8-methylnonanoic acid (tetrahydrofuran-3R-yl)amide
101 5-Amino-4-hydroxy-2-isopropyl-7-[4-methoxy-3-(3-methoxypropoxy)benzyl]-8-methylnonanoic acid (tetrahydrofuran-3S-yl)amide
102 5-Amino-4-hydroxy-2-isopropyl-7-[4-methoxy-3-(3-methoxypropoxy)benzyl]-8-methylnonanoic acid (cis-5-methoxytetrahydropyran-3-yl)amide

The starting materials are prepared as follows:

### a) cis-5-Methoxytetrahydropyran-3-ylamine

The title compound is prepared in analogy to Example 79 a from cis-5-methoxytetrahydropyran-3-carboxylic acid.

### b) cis-5-Methoxytetrahydropyran-3-carboxylic acid

A solution of 10.55 mmol of methyl cis-5-methoxytetrahydropyran-3-carboxylate in 45 ml of methanol is mixed with 45 ml of 1M LiOH and stirred at room temperature for one hour. The reaction mixture is evaporated and the residue is taken up in dichloromethane. The pH is adjusted to 2 with 2M HCl, the phases are separated, and the aqueous phase is extracted with dichloromethane (2×). The combined organic phases are dried with sodium sulphate and evaporated. The crude title compound is identified by means of its Rf from the residue.

### c) Methyl cis-5-methoxytetrahydropyran-3-carboxylate

15 ml of 50% NaOH, 367 mg of benzyltriethylammonium bromide and 0.95 ml of dimethyl sulphate are added to a solution of 1.5 g of methyl cis-5-hydroxytetrahydropyran-3-carboxylate in 30 ml of toluene. The two-phase mixture is stirred at room temperature for 18 hours. A further 1 ml of dimethyl sulphate is added. The mixture is stirred at room temperature for a further 6 hours and then diluted with water and ethyl acetate and stirred at room temperature for 20 minutes. The phases are separated and the aqueous phase is extracted with ethyl acetate. The combined organic phases are washed with brine, dried with sodium sulphate and evaporated. The title compound is obtained as a yellow oil from the residue by flash chromatography (SiO₂ 60 F) and identified by means of the Rf.

### d) Methyl cis-5-hydroxytetrahydropyran-3-carboxylate

A solution of diazomethane in diethyl ether (approx. 0.4 M) is added in excess to a solution of 23 mmol of cis-5-hydroxytetrahydropyran-3-carboxylic acid in 300 ml of methanol at 0° C. The mixture is stirred at 0° C. for 2 hours and then quenched with magnesium sulphate, filtered and evaporated. The crude title compound is identified by means of the Rf.

### e) cis-5-Hydroxytetrahydropyran-3-carboxylic acid

A solution of 25.5 mmol of cis-3,6-dioxabicyclo[3.2.1]octan-7-one in 500 ml of methanol is adjusted to pH 9 with 0.1N NaOH. The solution is stirred at constant pH until conversion is complete and is adjusted to pH 6.5 with 0.1N HCl before the methanol is evaporated off. The title compound is identified from the aqueous solution by means of the Rf by ion exchange chromatography (Amberlite XAD-2 resin).

### f) cis-3,6-Dioxabicyclo[3.2.1]octan-7-one and methyl trans-5-hydroxytetrahydropyran-3-carboxylate

A solution of 39 mmol of methyl 5-oxotetrahydropyran-3-carboxylate [127956-19-8] in 500 ml of methanol is cooled to 0° C., and 50 mmol of sodium borohydride are added in portions. The solution is stirred at 0° C. for 3 hours and quenched with water. It is neutralized with acetic acid, and water and tert-butyl methyl ether are added. The phases are separated and the aqueous phase is extracted with tert-butyl methyl ether (2×). The combined organic phases are washed with brine, dried with sodium sulphate and evaporated. The title compounds are identified by means of the Rf from the residue by flash chromatography (SiO₂ 60 F).
103 5-Amino-4-hydroxy-2-isopropyl-7-[4-methoxy-3-(3-methoxypropoxy)benzyl]-8-methylnonanoic acid (trans-5-methoxytetrahydropyran-3-yl)amide

The starting materials are prepared as follows:

### a) trans-5-Methoxytetrahydropyran-3-ylamine

The title compound is prepared in analogy to Example 79 a from trans-5-methoxytetrahydropyran-3-carboxylic acid.

### b) trans-5-Methoxytetrahydropyran-3-carboxylic acid

The title compound is prepared in analogy to Example 102b from methyl trans-5-methoxytetrahydropyran-3-carboxylate.

### c) Methyl trans-5-methoxytetrahydropyran-3-carboxylate

The title compound is prepared in analogy to Example 102c from methyl trans-5-hydroxytetrahydropyran-3-carboxylate (Example 102f).
104 5-Amino-4-hydroxy-2-isopropyl-7-[4-methoxy-3-(3-methoxypropoxy)benzyl]-8-methylnonanoic acid (cis-5-methoxytetrahydropyran-3-ylmethyl)amide

The starting material is prepared as follows:

### a) cis-C-(5-Methoxytetrahydropyran-3-yl)methylamine

The title compound is prepared in analogy to Example 65 a-c from cis-(5-methoxytetrahydropyran-3-yl)methanol.

### b) cis-(5-Methoxytetrahydropyran-3-yl)methanol

The title compound is prepared in analogy to Example 81c from methyl cis-5-methoxytetrahydropyran-3-carboxylate (Example 102c).
105 5-Amino-4-hydroxy-2-isopropyl-7-[4-methoxy-3-(3-methoxypropoxy)benzyl]-8-methylnonanoic acid (trans-5-methoxy-tetrahydropyran-3-ylmethyl)amide

The starting material is prepared as follows:

### a) trans-C-(5-Methoxytetrahydropyran-3-yl)methylamine

The title compound is prepared in analogy to Example 104 a-b from methyl trans-5-methoxytetrahydropyran-3-carboxylate (Example 103c).
106 5-Amino-4-hydroxy-2-isopropyl-7-[4-methoxy-3-(3-methoxypropoxy)benzyl]-8-methylnonanoic acid (cis-5-methoxyoxepan-3-yl)amide

The starting materials are prepared as follows:

### a) cis-5-Methoxyoxepan-3-ylamine

The title compound is prepared in analogy to Example 102 a-b from methyl cis-5-methoxyoxepane-3-carboxylate.

### b) Methyl cis-5-methoxyoxepane-3-carboxylate

The title compound is prepared in analogy to Example 102d-e from cis-3,7-dioxabicyclo[4.2.1]nonan-8-one.

### c) cis-3,7-Dioxabicyclo[4.2.1]nonan-8-one and methyl trans-5-hydroxyoxepane-3-carboxylate

The title compounds are prepared in analogy to Example 102f from methyl 5-oxooxepane-3-carboxylate [12756-13-2].
107 5-Amino-4-hydroxy-2-isopropyl-7-[4-methoxy-3-(3-methoxypropoxy)benzyl]-8-methylnonanoic acid (trans-5-methoxyoxepan-3-yl)amide

The starting materials are prepared as follows:

### a) trans-5-Methoxyoxepan-3-ylamine

The title compound is prepared in analogy to Example 103 a-b from methyl trans-5-methoxyoxepan-3-carboxylate.

### b) Methyl trans-5-methoxyoxepane-3-carboxylate

The title compound is prepared in analogy to Example 103c from methyl trans-5-hydroxyoxepane-3-carboxylate (Example 106c).
108 5-Amino-4-hydroxy-2-isopropyl-7-[4-methoxy-3-(3-methoxypropoxy)benzyl]-8-methylnonanoic acid (cis-5-methoxyoxepan-3-ylmethyl)amide

The starting material is prepared as follows:

### a) cis-C-(5-Methoxyoxepan-3-yl)methylamine

The title compound is prepared in analogy to Example 104 a-b from methyl cis-5-methoxyoxepane-3-carboxylate (Example 106b).
109 5-Amino-4-hydroxy-2-isopropyl-7-[4-methoxy-3-(3-methoxypropoxy)benzyl]-8-methylnonanoic acid (trans-5-methoxyoxepan-3-ylmethyl)amide

The starting material is prepared as follows:

### a) trans-C-(5-Methoxyoxepan-3-yl)methylamine

The title compound is prepared in analogy to Example 104 a-b from methyl trans-5-methoxyoxepane-3-carboxylate (Example 107b).
110 5-Amino-4-hydroxy-2-isopropyl-7-[4-methoxy-3-(3-methoxypropoxy)benzyl]-8-methylnonanoic acid (6R-methoxyoxepan-4-yl)amide

The starting material is prepared as follows:

### a) 6R-Methoxyoxepan-4-ylamine

2.8 mmol of sodium cyanoborohydride, 2.8 mmol of ammonium acetate and 530 mg of 4 Å molecular sieves are added to a solution of 2.8 mmol of 6R-methoxyoxepan-4-one in 11 ml of methanol. The reaction mixture is stirred for 18 hours, and conc. HCl is added until the pH falls below 3 and a white precipitate becomes visible. It is concentrated and, after addition of water, extracted with dichloromethane (1×). The aqueous phase is adjusted to pH 12 with 6M NaOH and extracted with dichloromethane (3×). The combined organic phases are washed with water, dried with sodium sulphate and evaporated. The title compound is identified by means of the Rf from the residue by flash chromatography (SiO₂ 60 F).

### b) 6R-Methoxyoxepan-4-one

The title compound is prepared in analogy to Example 85c from 3R-methoxy-5-methyleneoxepane.

### c) 3R-Methoxy-5-methyleneoxepane

The title compound is prepared in analogy to Example 102c from 3-hydroxy-5-methyleneoxepane [138907-09-2].
111 5-Amino-4-hydroxy-2-isopropyl-7-[4-methoxy-3-(3-methoxypropoxy)benzyl]-8-methylnonanoic acid (6-methoxy-oxepan-3-yl)amide

The starting material is prepared as follows:

### a) 6-Methoxyoxepan-3-ylamine

The title compound is prepared in analogy to Example 110 a-c from 6-hydroxyoxepan-3-one [120741-87-9].
112 5-Amino-4-hydroxy-2-isopropyl-7-[4-methoxy-3-(3-methoxypropoxy)benzyl]-8-methylnonanoic acid (4R-methoxytetrahydrofuran-2R-ylmethyl)amide

The starting material is prepared as follows:

### a) C-(4R-Methoxytetrahydrofuran-2R-yl)methylamine

The title compound is prepared in analogy to Example 65 a-c from (4R-methoxytetrahydrofuran-2R-yl)methanol.

### b) (4R-Methoxytetrahydrofuran-2R-yl)methanol

The title compound is prepared in analogy to Example 78 a from methyl 4R-Methoxyhydrofuran-2R-carboxylate.

### c) Methyl 4R-methoxytetrahydrofuran-2R-carboxylate

The title compound is prepared in analogy to Example 102c from methyl 4R-hydroxytetrahydrofuran-2R-carboxylate.

### d) Methyl 4R-hydroxytetrahydrofuran-2R-carboxylate

The title compound is prepared in analogy to Example 89d from methyl 4S-hydroxytetrahydrofuran-2R-carboxylate [2208-93-7].
113 5-Amino-4-hydroxy-2-isopropyl-7-[4-methoxy-3-(3-methoxypropoxy)benzyl]-8-methylnonanoic acid (4S-methoxytetrahydrofuran-2R-ylmethyl)amide

The starting material is prepared as follows:

### a) C-(4S-Methoxytetrahydrofuran-2R-yl)methylamine

The title compound is prepared in analogy to Example 112 a-c from methyl 4S-hydroxytetrahydrofuran-2R-carboxylate [2208-93-7].
114 5-Amino-4-hydroxy-2-isopropyl-7-[4-methoxy-3-(3-methoxypropoxy)benzyl]-8-methylnonanoic acid (4R-methoxytetrahydrofuran-2S-ylmethyl)amide

The starting material is prepared as follows:

### a) C-(4R-Methoxytetrahydrofuran-2S-yl)methylamine

The title compound is prepared in analogy to Example 112 a-c from methyl 4R-hydroxytetrahydrofuran-2S-carboxylate [2209-10-1].
115 5-Amino-4-hydroxy-2-isopropyl-7-[4-methoxy-3-(3-methoxypropoxy)benzyl]-8-methylnonanoic acid (4S-methoxytetrahydrofuran-2S-ylmethyl)amide

The starting material is prepared as follows:

### a) C-(4S-Methoxytetrahydrofuran-2S-yl)methylamine

The title compound is prepared in analogy to Example 112 a-c from methyl 4S-hydroxytetrahydrofuran-2S-carboxylate [2209-09-08].
117 5-Amino-4-hydroxy-2-isopropyl-7-[4-methoxy-3-(3-methoxypropoxy)benzyl]-8-methylnonanoic acid ((1S,2R,4S)-7-oxabicyclo[2.2.1]hept-2-yl)amide

The starting materials are prepared as follows:

### a) (1S,2R,4S)-7-Oxabicyclo[2.2.1]hept-2-ylamine

The title compound is prepared in analogy to Example 79 a from (1S,2R,4S)-7-oxabicyclo[2.2.1]heptane-2-carboxylic acid.

### b) (1S,2R,4S)-7-Oxabicyclo[2.2.1]heptane-2-carboxylic acid

A solution of 1 mmol of (1S,2R,4S)-7-oxabicyclo[2.2.1]hept-5-ene-2-carboxylic acid [185840-15-7] in 30 ml of ethanol is hydrogenated in the presence of 0.150 g of Pd/C 10% at room temperature until conversion is complete. The reaction mixture is clarified by filtration and the filtrate is evaporated. The title compound is obtained from the residue by flash chromatography (SiO₂ 60 F).
118 5-Amino-4-hydroxy-2-isopropyl-7-[4-methoxy-3-(3-methoxypropoxy)benzyl]-8-methylnonanoic acid ((1R,2S,4R)-7-oxabicyclo[2.2.1]hept-2-yl)amide

The starting materials are prepared as follows:

### a) (1R,2S,4R)-7-Oxabicyclo[2.2.1]hept-2-ylamine

The title compound is prepared in analogy to Example 117 a-b from (1R,2S,4R)-7-oxabicyclo[2.2.1]hept-5-ene-2-carboxylic acid.

### b) (1R,2S,4R)-7-Oxabicyclo[2.2.1]hept-5-ene-2-carboxylic acid

A solution of 1 mmol of (1R,2R)-2-(naphthalene-2-sulphonyl)cyclohexyl (exo)-(1R,2S,4R)-7-oxabicyclo[2.2.1]hept-5-ene-2-carboxylate in 5 ml of dimethylformamide is mixed with 2 mmol of potassium hydroxide and stirred at 60° C. for 17 hours. The reaction mixture is cooled to room temperature and, after addition of 1M citric acid solution, extracted with tert-butyl methyl ether (3×). The combined organic phases are washed with cold water and cold brine, dried with sodium sulphate and evaporated at room temperature. The title compound is identified by means of the Rf from the residue by flash chromatography (SiO₂ 60 F).
119 5-Amino-4-hydroxy-2-isopropyl-7-[4-methoxy-3-(3-methoxypropoxy)benzyl]-8-methylnonanoic acid ((1R,2R,4R)-7-oxabicyclo[2.2.1]hept-2-yl)amide

The starting material is prepared as follows:

### a) (1R,2R,4R)-7-Oxabicyclo[2.2.1]hept-2-ylamine

The title compound is prepared in analogy to Example 117 a-b from (1R,2R,4R)-7-oxabicyclo[2.2.1]hept-5-ene-2-carboxylic acid [90760-55-7].
120 5-Amino-4-hydroxy-2-isopropyl-7-[4-methoxy-3-(3-methoxypropoxy)benzyl]-8-methylnonanoic acid ((1S,2S,4S)-7-oxabicyclo[2.2.1]hept-2-yl)amide

The starting material is prepared as follows:

### a) (1S,2S,4S)-7-Oxabicyclo[2.2.1]hept-2-ylamine

The title compound is prepared in analogy to Example 117 a-b from (1S,2S,4S)-7-oxabicyclo[2.2.1]hept-5-ene-2-carboxylic acid [90760-56-8].
121 5-Amino-4-hydroxy-2-isopropyl-7-[4-methoxy-3-(3-methoxypropoxy)benzyl]-8-methylnonanoic acid (2-oxabicyclo[2.2.1]hept-6-yl)amide

The starting material is prepared as follows:

### a) 2-Oxabicyclo[2.2.1]hept-6-ylamine

The title compound is prepared in analogy to Example 89 a from 2-oxabicyclo[2.2.1]heptan-6-one [34108-25-3].
122 5-Amino-4-hydroxy-2-isopropyl-7-[4-methoxy-3-(3-methoxypropoxy)benzyl]-8-methylnonanoic acid ((exo)2,5-dioxabicyclo[4.1.0]hept-7-yl)amide

The starting material is prepared as follows:

### a) (exo)-(2,5-Dioxabicyclo[4.1.0]hept-7-yl)amine

The title compound is prepared in analogy to Example 79 a-b from (exo)-2,5-dioxabicyclo[4.1.0]heptane-7-carboxylic acid [60170-70-9].
123 5-Amino-4-hydroxy-2-isopropyl-7-[(S)-(3S,4S)-4-methoxy-3-(3-methoxypropoxy)-benzyl]-8-methylnonanoic acid [(exo)-1-(2,5-dioxabicyclo[4.1.0]hept-7-yl)methyl]amide

The starting material is prepared as follows:

### a) (exo)-(2,5-Dioxabicyclo[4.1.0]hept-7-ylmethyl)amine

The title compound is prepared in analogy to Example 77 a-b from ethyl (exo)-2,5-dioxabicyclo[4.1.0]heptane-7-carboxylate [60170-67-4].
124 (2S,4S,5S,7S)-5-Amino-4-hydroxy-2-isopropyl-7-[4-methoxy-3-(3-methoxypropoxy)benzyl]-8-methylnonanoic acid [(cis)-1-(3-oxabicyclo[3.1.0]hex-2-yl)methyl]amide

The starting material is prepared as follows:

### a) C-[(cis)-1-(3-Oxabicyclo[3.1.0]hex-2-yl)]methylamine

The title compound is prepared in analogy to Example 77 a-b and Example 88 from methyl (cis)-3-oxabicyclo[3.1.0]hexane-2-carboxylate.

### b) Methyl (cis)-3-oxabicyclo[3.1.0]hexane-2-carboxylate

The title compound is prepared in analogy to Example 102d from (cis)-3-oxabicyclo[3.1.0]hexane-2-carboxylic acid.

### c) (cis)-3-Oxabicyclo[3.1.0]hexane-2-carboxylic acid

The title compound is prepared in analogy to Example 88 from (cis)-3-oxabicyclo[3.1.0]hexane-2-methanol [85194-16-7].
127 (2S,4S,5S,7S)-5-Amino-4-hydroxy-2-isopropyl-7-[4-methoxy-3-(3-methoxypropoxy)-benzyl]-8-methylnonanoic acid [2-methyl-2-(tetrahydropyran-4-yl)propyl]amide

The starting material is prepared as follows:

### a) 2-Methyl-2-(tetrahydropyran-4-yl)propylamine

The title compound is prepared in analogy to Example 77 a-b from ethyl 2-methyl-2-(tetrahydropyran-4-yl)propionate [865156-84-9].
128 (2S,4S,5S,7S)-5-Amino-4-hydroxy-2-isopropyl-7-[4-methoxy-3-(3-methoxypropoxy)-benzyl]-8-methylnonanoic acid [2,2-difluoro-2-(tetrahydropyran-4-yl)ethyl]amide

The starting material is prepared as follows:

### a) 2,2-Difluoro-2-(tetrahydropyran-4-yl)ethylamine

The title compound is prepared in analogy to Example 77 a-b from methyl difluoro-(tetrahydropyran-4-yl)acetate.

### b) Methyl difluoro(tetrahydropyran-4-yl)acetate

The title compound is prepared in analogy to Example 102 d from difluoro(tetrahydropyran-4-yl)acetic acid.

### c) Difluoro(tetrahydropyran-4-yl)acetic acid

A solution of 0.774 mmol of 2-[1,1-dichloro-2,2-difluoro-2-(tetrahydropyran-4-yl)ethylsulphanyl]pyridine in 4 ml of tetrahydrofuran is mixed with 3.096 mmol of silver nitrate solution (in 4 ml of water). The cloudy mixture is heated to reflux for 3 hours. The reaction mixture is cooled to room temperature and filtered through Hyflow. The filtrate is adjusted to pH 9-10 with 50% strength sodium bicarbonate solution and washed with diethyl ether. The aqueous phase is subsequently adjusted to pH 1 with 50% strength H₂SO₄ and extracted with dichloromethane. The combined organic phases are dried with sodium sulphate and evaporated. The title compound is obtained as a yellow solid from the residue.

### d) 2-[1,1-Dichloro-2,2-difluoro-2-(tetrahydropyran-4-yl)ethylsulphanyl]pyridine

2.422 mmol of N,N′-dicyclohexylcarbodiimide are added to a solution of 2.422 mmol of tetrahydropyran-4-carboxylic acid [5337-03-1] and 2.422 mmol of hydroxythiopyrimidone [1121-31-9] in 5 ml of dichloromethane at room temperature with exclusion of light. The reaction mixture is stirred at room temperature for 3 hours. It is filtered through Hyflow, and the filtrate is evaporated with exclusion of light. The yellow residue is dissolved in 4 ml of acetonitrile, the solution is cooled to 0° C., and 24.22 mmol of 1,1-dichloro-2,2-difluoroethylene [79-35-6] are added. The reaction mixture is stirred under the influence of light (300 W sunlamp) at −10° C. for 2.5 hours. It is evaporated. The title compound is obtained as a pale yellow oil from the residue by flash chromatography (SiO₂ 60 F). Rf=0.26 (EtOAc/heptane 1:1); Rt=4.25 (gradient I).

### Example 30

### 5-Amino-4-hydroxy-2-isopropyl-7-[3-(3-methoxypropoxy)-4-methylbenzyl]-8-methylnonanoic acid pyrrolidin-3(S)-ylamide

0.35 g of tert-butyl 3(S)-{5-tert-butoxycarbonylamino-4-hydroxy-2-isopropyl-7-[3-(3-methoxypropoxy)-4-methylbenzyl]-8-methylnonanoylamino}pyrrolidine-1-carboxylate is reacted in analogy to Method K. The title compound is obtained as a white foam. Rf=0.13 (dichloromethane/methanol/conc. ammonia 25% 40:10:1); Rt=3.56 (gradient I).

The starting materials are prepared as follows:

### a) tert-Butyl 3(S)-{5-tert-butoxycarbonylamino-4-hydroxy-2-isopropyl-7-[3-(3-methoxypropoxy)-4-methylbenzyl]-8-methylnonanoylamino}pyrrolidine-1-carboxylate

0.400 g of tert-butyl {1-(4-isopropyl-5-oxotetrahydrofuran-2-yl)-3-[3-(3-methoxypropoxy)-4-methylbenzyl]-4-methylpentyl}carbamate is reacted with tert-butyl (S)-3-aminopyrrolidine-1-carboxylate [147081-44-5] in analogy to Method B. The title compound is obtained as a white solid. Rf=0.12 (EtOAc/heptane 1:1); Rt=5.66 (gradient I).

### b) tert-Butyl {1-(4-isopropyl-5-oxotetrahydrofuran-2-yl)-3-[3-(3-methoxpropoxy)-4-methylbenzyl]-4-methylpentyl}carbamate

5.8 g of 5-{1-amino-3-[3-(3-methoxypropoxy)-4-methylbenzyl]-4-methylpentyl}-3-isopropyl-dihydrofuran-2-one are reacted in analogy to Method J. The title compound is obtained as a beige oil. Rf=0.31 (EtOAc/heptane 1:3); Rt=6.16 (gradient I).

### c) 5-{1-Amino-3-[3-(3-methoxypropoxy)-4-methylbenzyl]-4-methylpentyl}-3-isopropyl-dihydrofuran-2-one

8.0 g of 2-[2-azido-2-(4-isopropyl-5-oxotetrahydrofuran-2-yl)ethyl]-1-[3-(3-methoxypropoxy)-4-methylphenyl]-3-methylbutyl methoxyacetate are reacted in analogy to Method I. The title compound is obtained as a yellow oil. Rf=0.05 (EtOAc/heptane 1:1); Rt=4.55 (gradient I).

### d) 2-[2-Azido-2-(4-isopropyl-5-oxotetrahydrofuran-2-yl)ethyl]-1-[3-(3-methoxypropoxy)-4-methylphenyl]-3-methylbutyl methoxyacetate

8.0 g of 5-(1-azido-3-{hydroxy-[3-(3-methoxypropoxy)-4-methylphenyl]methyl}-4-methylpentyl)-3-isopropyldihydrofuran-2-one are reacted in analogy to Method H. The title compound is obtained as a yellow oil. Rf=0.17 (EtOAc/heptane 1:2); Rt=5.70 (gradient I).

### e) 5-(1-Azido-3-{hydroxy-[3-(3-methoxypropoxy)-4-methylphenyl]methyl}-4-methylpentyl)-3-isopropyldihydrofuran-2-one

6.0 g of 4-bromo-2-(3-methoxypropoxy)-1-methylbenzene are reacted in analogy to Method G. The title compound is obtained as a beige oil. Rf=0.38 (EtOAc/heptane 1:1); Rt=5.29 and 5.57 (gradient I).

### f) 4-Bromo-2-(3-methoxypropoxy)-1-methylbenzene

6.0 g of 4-bromo-1-methylphenol [2362-12-1] are reacted in analogy to Method L. The title compound is obtained as an orange oil. Rf=0.38 (EtOAc/heptane 1:10); Rt=5.37 (gradient I).

The following compounds are prepared in an analogous manner to the process described in Example 30:
31 5-Amino-4-hydroxy-2-isopropyl-7-[3-(3-methoxypropoxy)-4-methylbenzyl]-8-methylnonanoic acid pyrrolidin-3(R)-ylamide

White foam; Rf=0.11 (dichloromethane/methanol/conc. ammonia 25% 40:10:1); Rt=3.63 (gradient I).
40 5-Amino-4-hydroxy-2-isopropyl-7-[3-(3-methoxypropoxy)-4-methylbenzyl]-8-methylnonanoic acid (tetrahydrofuran-2(S)-ylmethyl)amide

Yellowish oil; Rf=0.52 (dichloromethane/methanol/conc. ammonia 25% 200:20:1); Rt=4.27 (gradient I).
34 5-Amino-4-hydroxy-2-isopropyl-7-[4-methoxy-3-(4-methoxybutyl)benzyl]-8-methylnonanoic acid pyrrolidin-3(S)-ylamide

Colourless resin; Rf=0.06 (dichloromethane/methanol/conc. ammonia 25% 30:20:1); Rt=3.53 (gradient I).

The starting materials are prepared as follows:

### a) 4-Bromo-1-methoxy-2-(4-methoxybutyl)benzene

A solution of 0.435 mg of 4-bromo-1-methoxy-2-(4-methoxybut-1-enyl)benzene in 23 ml of ethyl acetate at 0° C. is mixed with 92 μl of glacial acetic acid and hydrogenated in the presence of 600 mg of Pd/C 10% (moist) at 0° C. for 1.2 hours. The reaction mixture is clarified by filtration and the catalyst is washed with ethanol. The filtrate is evaporated. The residue is treated with 1M sodium bicarbonate solution and extracted with tert-butyl methyl ether (3×)—the combined organic phases are dried with sodium sulphate and evaporated. The title compound is obtained as a yellow oil from the residue by flash chromatography (SiO₂ 60 F). Rf=0.38 (EtOAc/heptane 1:4); Rt=5.35 (gradient I).

### b) 4-Bromo-1-methoxy-2-(4-methoxybut-1-enyl)benzene

A solution of 1.6 g of (3-methoxypropyl)triphenylphosphonium iodide [133622-76-1] in 7.5 ml of tetrahydrofuran at 0° C. is mixed with 3.5 ml of sodium bis(trimethylsilyl)amide solution (1M in tetrahydrofuran) and stirred at 0° C. for 30 minutes. Then 500 mg of 5-bromo-2-methoxybenzaldehyde are added, and the reaction mixture is warmed to room temperature. After 1 hour, it is diluted with 1M sodium bicarbonate solution and tert-butyl methyl ether. The phases are separated, and the organic phase is washed with 1M sodium bicarbonate solution, dried with sodium sulphate and evaporated. The title compound is obtained as an orange oil from the residue by flash chromatography (SiO₂ 60 F). Rf=0.36 (EtOAc/heptane 1:4); Rt=5.10 (gradient I).
35 5-Amino-4-hydroxy-2-isopropyl-7-[4-methoxy-3-(4-methoxybutyl)benzyl]-8-methylnonanoic acid pyrrolidin-3(R)-ylamide

Yellow resin; Rf=0.06 (dichloromethane/methanol/conc. ammonia 25% 30:20:1); Rt=3.62 (gradient I).
43 5-Amino-4-hydroxy-2-isopropyl-7-[4-methoxy-3-(4-methoxybutyl)benzyl]-8-methylnonanoic acid (tetrahydrofuran-2(S)-ylmethyl)amide

Yellow oil; Rf=0.27 (dichloromethane/methanol/conc. ammonia 25% 200:20:1); Rt=4.44 (gradient I).
41 5-Amino-7-[3-ethyl-4-(3-methoxypropoxy)benzyl]-4-hydroxy-2-isopropyl-8-methylnonanoic acid (tetrahydrofuran-2(S)-ylmethyl)amide

Yellowish oil; Rf=0.43 (dichloromethane/methanol/conc. ammonia 25% 200:20:1); Rt=4.41 (gradient I).

The starting material is prepared as follows:

### a) 4-Bromo-2-ethyl-1-(3-methoxypropoxy)benzene

17.6 g of 4-bromo-2-ethylphenol [18980-21-7] are reacted in analogy to Method L. The title compound is obtained as a colourless oil. Rf=0.38 (EtOAc/heptane 1:4); Rt=5.64 (gradient I).
42 5-Amino-7-[3-ethyl-4-(3-methoxypropoxy)benzyl]-4-hydroxy-2-isopropyl-8-methylnonanoic acid (tetrahydropyran-4-yl)amide

Yellowish oil; Rf=0.38 (dichloromethane/methanol/conc. ammonia 25% 200:20:1); Rt=4.20 (gradient I).
51 5-Amino-7-[4-ethyl-3-(3-methoxypropoxy)benzyl]-4-hydroxy-2-isopropyl-8-methylnonanoic acid (tetrahydropyran-4-yl)amide

Yellowish oil; Rf=0.45 (dichloromethane/methanol/conc. ammonia 25% 200:20:1); Rt=4.34 (gradient I).

The starting material is prepared as follows:

### a) 5-Bromo-2-ethyl-1-(3-methoxypropoxy)benzene

19.1 g of 5-bromo-2-ethylphenol [56152-25-1] are reacted in analogy to Method L. The title compound is obtained as a colourless liquid. Rf=0.55 (EtOAc/heptane 1:4); Rt=5.66 (gradient I).
69 5-Amino-7-[4-ethyl-3-(3-methoxypropoxy)benzyl]-4-hydroxy-2-isopropyl-8-methylnonanoic acid (tetrahydrofuran-2(S)-ylmethyl)amide
52 5-Amino-7-[4-cyclopropyl-3-(3-methoxypropoxy)benzyl]-4-hydroxy-2-isopropyl-8-methylnonanoic acid (tetrahydropyran-4-yl)amide

Yellow oil; Rf=0.21 (dichloromethane/methanol/conc. ammonia 25% 200:20:1); Rt=4.29 (gradient I).

The starting materials are prepared as follows:

### a) 4-Bromo-1-cyclopropyl-2-(3-methoxypropoxy)benzene

A solution of 164 ml of diethyl zinc solution (1M in hexane) in 224 ml of dichloromethane at 0° C. is mixed with a solution of 12.5 ml of trifluoroacetic acid in 111 ml of dichloromethane and stirred at 0° C. for 20 minutes. Then a solution of 13.2 ml of diiodomethane in 111 ml of dichloromethane is added, and the mixture is stirred at 0° C. for 20 minutes. A solution of 22.3 g of 4-bromo-2-(3-methoxypropoxy)-1-vinylbenzene in 111 ml of dichloromethane is added, and the mixture is warmed to room temperature and stirred for 30 minutes. The reaction mixture is quenched with saturated ammonium chloride solution, and the phases are separated. The aqueous phase is extracted with dichloromethane (2×)—the combined organic phases are washed with saturated sodium bicarbonate solution and brine, dried with sodium sulphate and evaporated. The title compound is obtained as a yellow oil from the residue by flash chromatography (SiO₂ 60 F). Rf=0.40 (EtOAc/heptane 1:6); Rt=5.50 (gradient I).

### b) 4-Bromo-2-(3-methoxypropoxy)-1-vinyl benzene

A solution of 27.7 g of 1-[4-bromo-2-(3-methoxypropoxy)phenyl]ethanol in 1 l of dimethyl sulphoxide is mixed with 13.18 g of potassium hydrogensulphate. The reaction mixture is stirred at 180° C. (preheated oil bath) for 2.5 hours and cooled to room temperature. The reaction mixture is partitioned between tert-butyl methyl ether and water. The aqueous phase is extracted with tert-butyl methyl ether (2×)—the combined organic phases are dried with sodium sulphate and evaporated. The title compound is obtained as a yellow oil from the residue by flash chromatography (SiO₂ 60 F). Rf=0.25 (EtOAc/heptane 1:6); Rt=5.38 (gradient I).

### c) 1-[4-Bromo-2-(3-methoxypropoxy)phenyl]ethanol

200 mg of sodium borohydride are added in portions to a solution of 1.12 g of 1-[4-bromo-2-(3-methoxypropoxy)phenyl]ethanone in 50 ml of ethanol at 0° C. The reaction mixture is warmed to room temperature and stirred at room temperature overnight. The reaction mixture is poured into ice-water and stirred for 15 minutes. The solution is adjusted to pH 5 with glacial acetic acid. The resulting solution is extracted with tert-butyl methyl ether (3×)—the combined organic phases are washed with brine, dried with sodium sulphate and evaporated. The title compound is obtained as a colourless oil from the residue by flash chromatography (SiO₂ 60 F). Rf=0.45 (EtOAc/heptane 1:1); Rt=4.16 (gradient I).

### d) 1-[4-Bromo-2-(3-methoxypropoxy)phenyl]ethanone

50 g of 1-(4-bromo-2-hydroxyphenyl)ethanone [30186-18-6] are reacted in analogy to Method L. The title compound is obtained as a yellow oil. Rf=0.30 (EtOAc/heptane 1:2); Rt=4.59 (gradient I).
70 5-Amino-7-[4-cyclopropyl-3-(3-methoxypropoxy)benzyl]-4-hydroxy-2-isopropyl-8-methylnonanoic acid (tetrahydrofuran-2(S)-ylmethyl)amide
56 5-Amino-4-hydroxy-2-isopropyl-7-[3-(3-methoxypropoxy)-4-trifluoromethoxybenzyl]-8-methylnonanoic acid (tetrahydropyran-4-yl)amide

The starting materials are prepared as follows:

### a) 4-Bromo-2-(3-methoxypropoxy)-1-trifluoromethoxybenzene

20 mmol of 1-(4-bromo-2-hydroxyphenyl)ethanone [30186-18-6] are reacted in analogy to Method L. The title compound is identified by means of its Rf.

### b) 5-Bromo-2-trifluoromethoxyphenol

A solution of 210 mmol of sodium nitrite in 120 ml of water is added dropwise to a suspension of 200 mmol of 5-bromo-2-trifluoromethoxyaniline in 500 ml of ethanol and 50 ml of conc. HCl at 0° C. The reaction mixture is stirred at 5° C. for 1.5 hours. The reaction mixture is slowly added dropwise to a solution of 135 ml of conc. sulphuric acid in 2.8 l of water and stirred under reflux overnight. The reaction mixture is extracted with diethyl ether (3×)—the combined organic phases are washed with water and 1M sodium bicarbonate solution and then extracted with 2N NaOH (2×). The combined aqueous phases are acidified with conc. HCl and extracted with diethyl ether (3×). The combined organic phases are washed with water, dried with sodium sulphate and evaporated. The crude title compound is obtained from the residue.
57 5-Amino-4-hydroxy-2-isopropyl-7-[3-(3-methoxypropoxy)-4-trifluoromethylbenzyl]-8-methylnonanoic acid (tetrahydropyran-4-yl)amide

The starting material is prepared as follows:

### a) 4-Bromo-2-(3-methoxypropoxy)-1-trifluoromethylbenzene

10.4 mmol of sodium hydride (60% dispersion in oil) are added in portions to a solution of 8.7 mmol of 3-methoxypropanol [1589-49-7] in 12 ml of dimethyl sulphoxide. The reaction mixture is stirred at room temperature for 30 minutes, and 1.3 g of sodium benzoate are added. The mixture is stirred at room temperature for 30 minutes, and 10.6 mmol of 4-bromo-2-fluoro-1-trifluoromethylbenzene [1428808-15-9] are added in such a way that the internal temperature does not exceed 20° C. After the addition is complete, the mixture is heated at 65° C. for 15 hours. It is then cooled to room temperature. The reaction mixture is quenched with water/brine 1:1 and extracted with dichloromethane (2×)—the combined organic phases are washed with brine, dried with sodium sulphate and evaporated. The title compound is identified by means of its Rf from the residue by flash chromatography (SiO₂ 60 F).
72 5-Amino-4-hydroxy-2-isopropyl-7-[3-(3-methoxypropoxy)-4-trifluoromethylbenzyl]-8-methylnonanoic acid (tetrahydrofuran-2(S)-ylmethyl)amide
91 5-Amino-7-[4-fluoro-3-(3-methoxypropoxy)benzyl]-4-hydroxy-2-isopropyl-8-methylnonanoic acid (tetrahydrofuran-2(S)-ylmethyl)amide

The starting material is prepared as follows:

### a) 4-Bromo-1-fluoro-2-(3-methoxypropoxy)benzene

30.22 g of 5-bromo-2-fluorophenol [112204-58-7] are reacted in analogy to Method L. The title compound is identified by means of its Rf.
92 5-Amino-7-[4-fluoro-3-(3-methoxypropoxy)benzyl]-4-hydroxy-2-isopropyl-8-methylnonanoic acid (tetrahydropyran-4-yl)amide
93 5-Amino-7-[4-fluoro-3-(3-methoxypropoxy)benzyl]-4-hydroxy-2-isopropyl-8-methylnonanoic acid (tetrahydropyran-4-ylmethyl)amide
94 5-Amino-7-[4-fluoro-3-(3-methoxypropoxy)benzyl]-4-hydroxy-2-isopropyl-8-methylnonanoic acid ((exo,endo)-3-oxabicyclo[3.1.0]hex-6-ylmethyl)amide

The starting material is prepared as described in Example 78.
95 5-Amino-7-[4-fluoro-3-(3-methoxypropoxy)benzyl]-4-hydroxy-2-isopropyl-8-methylnonanoic acid ((Z)-2-oxabicyclo[3.1.0]hex-1-ylmethyl)amide

The starting material is prepared as described in Example 85.

### Example 32

### 5-Amino-4-hydroxy-2-isopropyl-7-[3-(3-methoxypropoxy)-4-methylbenzyl]-8-methylnonanoic acid Piperidin-4-ylamide

220 mg of tert-butyl [2-hydroxy-1-{2-[3-(3-methoxypropoxy)-4-methylbenzyl]-3-methylbutyl}-5-methyl-4-(piperidin-4-ylcarbamoyl)hexyl]carbamate are reacted in analogy to Method K. The title compound is obtained as a white foam. Rf=0.13 (dichloromethane/methanol/conc. ammonia 25% 40:10:1); Rt=3.55 (gradient I).

The starting materials are prepared as follows:

### a) tert-Butyl [2-hydroxy-1-{2-[3-(3-methoxypropoxy)-4-methylbenzyl]-3-methylbutyl}-5-methyl-4-(piperidin-4-ylcarbamoyl)hexyl]carbamate

A solution of 358 mg of benzyl 4-{5-tert-butoxycarbonylamino-4-hydroxy-2-isopropyl-7-[3-(3-methoxypropoxy)-4-methylbenzyl]-8-methylnonanoylamino}piperidin-1-carboxylate in 15 ml of ethanol and 0.03 ml of ethanolamine is hydrogenated in the presence of 152 mg of Pd/C 10% (moist) at 0° C. for 1 hour. The reaction mixture is clarified by filtration and the catalyst is washed with ethanol. The filtrate is evaporated. The title compound is obtained as a white foam from the residue by flash chromatography (SiO₂ 60 F). Rf=0.1 (dichloromethane/methanol/conc. ammonia 25% 200:20:1); Rt=4.74 (gradient I).

### b) Benzyl 4-{5-tert-butoxycarbonylamino-4-hydroxy-2-isopropyl-7-[3-(3-methoxypropoxy)-4-methylbenzyl]-8-methylnonanoylamino}piperidine-1-carboxylate

0.400 g of tert-butyl {1-(4-isopropyl-5-oxotetrahydrofuran-2-yl)-3-[3-(3-methoxypropoxy)-4-methylbenzyl]-4-methylpentyl}carbamate (Example 30b) is reacted with benzyl 4-aminopiperidine-1-carboxylate [120278-07-1] in analogy to Method B. The title compound is obtained as a white foam. Rf=0.18 (EtOAc/heptane 1:1); Rt=5.72 (gradient I).

The following compound is prepared in an analogous manner to the process described in Example 32:
33 5-Amino-4-hydroxy-2-isopropyl-7-[4-methoxy-3-(4-methoxybutyl)benzyl]-8-methylnonanoic acid piperidin-4-ylamide

Yellowish foam; Rf=0.06 (dichloromethane/methanol/conc. ammonia 25% 50:10:1); Rt=3.55 (gradient I).

### Example 45

### 5-Amino-7-[4-(1,1-difluoroethyl)-3-(3-methoxypropoxy)benzyl]-4-hydroxy-2-isopropyl-8-methylnonanoic acid (tetrahydropyran-4-yl)amide

3.6 ml of HCl (1M in dioxane) are added to 360 mg of tert-butyl [1-{2-[4-(1,1-difluoroethyl)-3-(3-methoxypropoxy)benzyl]-3-methylbutyl}-2-hydroxy-5-methyl-4-(tetrahydropyran-4-ylcarbamoyl)hexyl]carbamate at 0° C. The reaction mixture is poured into ice/1M sodium bicarbonate solution and extracted with tert-butyl methyl ether (3×)—the combined organic phases are dried with sodium sulphate and evaporated. The title compound is obtained as a white foam from the residue by flash chromatography (SiO₂ 60 F). Rf=0.17 (dichloromethane/methanol/conc. ammonia 25% 200:20:1); Rt=4.14 (gradient I).

The starting materials are prepared as follows:

### a) tert-Butyl [1-{2-[4-(1,1-difluoroethyl)-3-(3-methoxypropoxy)benzyl]-3-methylbutyl}-2-hydroxy-5-methyl-4-(tetrahydropyran-4-ylcarbamoyl)hexyl]carbamate

The title compound is obtained as a white foam in analogy to Example 30, sequence a-e, from 4-bromo-1-(1,1-difluoroethyl)-2-(3-methoxypropoxy)benzene. Rf=0.19 (EtOAc/heptane 2:1); Rt=5.33 (gradient I).

### b) 4-Bromo-1-(1,1-difluoroethyl)-2-(3-methoxypropoxy)benzene

A solution of 15.7 g of 1-[4-bromo-2-(3-methoxypropoxy)phenyl]ethanone (Example 52 d) in 2.78 ml of Deoxofluor® in a Teflon vessel is stirred at 85° C. for 2 days. The reaction mixture is poured into cold 1M sodium bicarbonate solution and extracted with dichloromethane (2×)—the combined organic phases are dried with sodium sulphate and evaporated. The title compound is obtained as a yellow liquid from the residue by flash chromatography (SiO₂ 60 F). Rf=0.67 (EtOAc/heptane 1:1); Rt=5.02 (gradient I).

The following compound is prepared in an analogous manner to the process described in Example 45:
47 5-Amino-7-[4-(1,1-difluoroethyl)-3-(3-methoxypropoxy)benzyl]-4-hydroxy-2-isopropyl-8-methylnonanoic acid (tetrahydrofuran-2(S)-ylmethyl)amide

Yellowish oil; Rf=0.22 (dichloromethane/methanol/conc. ammonia 25% 200:20:1); Rt=4.32 (gradient I).

### Example 46

### 7-[4-Acetyl-3-(3-methoxypropoxy)benzyl]-5-amino-4-hydroxy-2-isopropyl-8-methylnonanoic acid (tetrahydropyran-4-yl)amide

0.42 g of tert-butyl [1-{2-[4-(1,1-difluoroethyl)-3-(3-methoxypropoxy)benzyl]-3-methylbutyl}-2-hydroxy-5-methyl-4-(tetrahydropyran-4-ylcarbamoyl)hexyl]carbamate (Example 45 a) is reacted in analogy to Method K. The title compound is obtained as a yellow oil. Rf=0.16 (dichloromethane/methanol/conc. ammonia 25% 200:20:1); Rt=3.59 (gradient I).

The following compound is prepared in an analogous manner to the process described in Example 46:
48 7-[4-Acetyl-3-(3-methoxypropoxy)benzyl]-5-amino-4-hydroxy-2-isopropyl-8-methylnonanoic acid (tetrahydrofuran-2(S)-ylmethyl)amide

Yellowish oil; Rf=0.25 (dichloromethane/methanol/conc. ammonia 25% 200:20:1); Rt=3.76 (gradient I).

### Example 53

### 5-Amino-7-[4-chloro-3-(3-methoxypropoxy)benzyl]-4-hydroxy-2-isopropyl-8-methylnonanoic acid (tetrahydropyran-4-yl)amide

30 mg of tert-butyl [1-{2-[4-chloro-3-(3-methoxypropoxy)benzyl]-3-methylbutyl}-2-hydroxy-5-methyl-4-(tetrahydropyran-4-ylcarbamoyl)hexyl]carbamate are reacted in analogy to Method K. The title compound is obtained as a colourless glass. Rf=0.34 (dichloromethane/methanol/conc. ammonia 25% 200:20:1); Rt=4.02 (gradient I).

The starting materials are prepared as follows:

### a) tert-Butyl [1-{2-[4-chloro-3-(3-methoxypropoxy)benzyl]-3-methylbutyl}-2-hydroxy-5-methyl-4-(tetrahydropyran-4-ylcarbamoyl)hexyl]carbamate

63 mg of tert-butyl [3-[4-chloro-3-(3-methoxypropoxy)benzyl]-1-(4-isopropyl-5-oxotetrahydrofuran-2-yl)-4-methylpentyl]carbamate are reacted in analogy to Method B. The title compound is obtained as a colourless oil. Rf=0.15 (EtOAc/heptane 2:1); Rt=5.16 (gradient I).

### b) tert-Butyl [3-[4-chloro-3-(3-methoxypropoxy)benzyl]-1-(4-isopropyl-5-oxotetrahydrofuran-2-yl)-4-methylpentyl]carbamate

Tributyltin hydride (0.36 ml) is added to a degassed solution of 0.676 g of O-{2-[2-tert-butoxycarbonylamine-2-isopropyl-5-oxotetrahydrofuran-2-yl)ethyl-1-[4-chloro-3-(3-methoxypropoxy)phenyl]-3-methylbutyl imidazolecarbothioate and 0.0271 g of 2,2′-azobisisobutyronitrile in 15 ml of toluene. The flask is placed in an oil bath preheated to 120°, and the reaction solution is stirred under reflux for 3 hours. The reaction mixture is then cooled to room temperature, diluted with ethyl acetate and washed with 0.1M HCl and brine, dried with sodium sulphate and evaporated. The title compound is obtained as a colourless oil from the residue by flash chromatography (SiO₂ 60 F). Rf=0.33 (EtOAc/heptane 1:2), Rt=5.82 (gradient I).

### c) O-{2-[2-tert-Butoxycarbonylamine-2-isopropyl-5-oxotetrahydrofuran-2-yl)ethyl-1-[4-chloro-3-(3-methoxypropoxy)phenyl]-3-methylbutyl imidazolecarbothioate

A solution of 0.847 g of tert-butyl [3-{[4-chloro-3-(3-methoxypropoxy)phenyl]hydroxymethyl}-1-(4-isopropyl-5-oxotetrahydrofuran-2-yl)-4-methylpentyl]carbamate, 0.993 g of 1,1′-thiocarbonyldiimidazole and 0.019 g of 4-dimethylaminopyridine in 5 ml of tetrahydrofuran is heated to reflux for 3 hours. A further portion of 1,1′-thiocarbonyldiimidazole (0.300 g) is added and then heating to reflux is continued for 3 hours. The reaction mixture is cooled to room temperature, diluted with ethyl acetate, washed with brine, dried with sodium sulphate and evaporated. The title compound is obtained as a yellow foam from the residue by flash chromatography (SiO₂ 60 F). Rf=0.41 (EtOAc/heptane 1:1), Rt=5.53 (gradient I).

### d) tert-Butyl [3-{[4-chloro-3-(3-methoxypropoxy)phenyl]hydroxymethyl}-1-(4-isopropyl-5-oxotetrahydrofuran-2-yl)-4-methylpentyl]carbamate

A solution of 1.311 g of 5-(1-azido-3-{[4-chloro-3-(3-methoxypropoxy)phenyl]hydroxymethyl}-4-methylpentyl)-3-isopropyldihydrofuran-2-one in 15 ml of tetrahydrofuran is mixed with 0.865 g of triphenylphosphine and 0.064 ml of water. The reaction solution is stirred at room temperature for 16 hours. Water (0.40 ml) is added, and the reaction mixture is then heated to reflux for 8 hours. The reaction mixture is cooled to room temperature, diluted with tert-butyl methyl ether (100 ml) and washed with brine (50 ml), dried with sodium sulphate and evaporated. The residue is dissolved in 20 ml of tetrahydrofuran and, after addition of 0.713 ml of Hünig's base and 0.727 g of di-tert-butyl dicarbonate, left to stand at room temperature for 12 hours. The reaction solution is diluted with tert-butyl methyl ether (50 ml) and washed with 0.1M HCl and brine, dried with sodium sulphate and evaporated. The title compound is obtained as a colourless glass from the residue by flash chromatography (SiO₂ 60 F). Rf=0.42 (EtOAc/heptane 1:1), Rt=5.26 (gradient I).

### e) 5-(1-Azido-3-{[4-chloro-3-(3-methoxypropoxy)phenyl]hydroxymethyl}-4-methylpentyl)-3-isopropyldihydrofuran-2-one

7 g of 4-bromo-1-chloro-2-(3-methoxypropoxy)benzene are reacted in analogy to Method G. The title compound is obtained as a colourless oil. Rf=0.52 (EtOAc/heptane 1:1), Rt=5.27 (gradient I).

### f) 4-Bromo-1-chloro-2-(3-methoxypropoxy)benzene

30.22 g of 5-bromo-2-chlorophenol [183802-98-4] are reacted in analogy to Method L. The title compound is obtained as a yellowish oil. Rf=0.40 (EtOAc/heptane 1:4), Rt=5.00 (gradient I).

The following compound is prepared in an analogous manner to the process described in Example 53:
71 5-Amino-7-[4-chloro-3-(3-methoxypropoxy)benzyl]-4-hydroxy-2-isopropyl-8-methylnonanoic acid (tetrahydrofuran-2(S)-ylmethyl)amide

### Example 73

### 5-Amino-4-hydroxy-2-isopropyl-7-[3-(4-methoxybutyryl)-4-methylbenzyl]-8-methylnonanoic acid (tetrahydropyran-4-yl)amide

0.5 mmol of tert-butyl [2-hydroxy-1-{2-[3-(4-methoxybutyryl)-4-methylbenzyl]-3-methylbutyl}-5-methyl-4-(tetrahydropyran-4-ylcarbamoyl)hexyl]carbamate is reacted in analogy to Method K. The title compound is identified by means of its Rf.

The starting materials are prepared as follows:

### a) tert-Butyl [2-hydroxy-1-{2-[3-(4-methoxybutyryl)-4-methylbenzyl]-3-methylbutyl}-5-methyl-4-(tetrahydropyran-4-ylcarbamoyl)hexyl]carbamate

The title compound is obtained in analogy to Example 30, sequence a-d, from 5-(1-azido-3-{hydroxy[3-(4-methoxybutyryl)-4-methylphenyl]methyl}-4-methylpentyl)-3-isopropyldihydrofuran-2-one and identified by means of its Rf.

### b) 5-(1-Azido-3-{hydroxy[3-(4-methoxybutyryl)-4-methylphenyl}methyl]-4-methylpentyl)-3-isopropyldihydrofuran-2-one

A solution of 0.62 mmol of 5-[1-azido-3-(hydroxy-{3-[2-(3-methoxypropyl)-[1,3]dioxolan-2-yl]-4-methylphenyl}methyl)-4-methylpentyl]-3-isopropyldihydrofuran-2-one in 20 ml of acetone is mixed with 20 ml of 2% HCl. The reaction mixture is stirred at room temperature for 45 minutes and poured into saturated sodium bicarbonate solution. It is extracted with dichloromethane (2×)—the combined organic phases are dried with sodium sulphate and evaporated. The title compound is identified by means of its Rf from the residue by flash chromatography (SiO₂ 60 F).

### c) 5-[1-Azido-3-(hydroxy-{3-[2-(3-methoxypropyl)-[1,3]dioxolan-2-yl]-4-methylphenyl}methyl)-4-methylpentyl]-3-isopropyldihydrofuran-2-one

1 mmol of 2-(5-bromo-2-methylphenyl)-2-(3-methoxypropyl)-[1,3]dioxolane is reacted in analogy to Method G. The title compound is identified by means of its Rf.

### d) 2-(5-Bromo-2-methylphenyl)-2-(3-methoxypropyl)-[1,3]dioxolane

A solution of 6.76 mmol of 1-(5-bromo-2-methylphenyl)-4-methoxybutan-1-one in 100 ml of ethylene glycol is mixed with 1 ml of glacial acetic acid. The reaction mixture is stirred at room temperature for 16 hours and poured into saturated sodium bicarbonate solution. It is extracted with dichloromethane (2×)—the combined organic phases are dried with sodium sulphate and evaporated. The title compound is identified by means of its Rf from the residue by flash chromatography (SiO₂ 60 F).

### e) 1-(5-Bromo-2-methylphenyl)-4-methoxybutan-1-one

1.05 mol of 4-methoxybutanoyl chloride [61882-39-1] are added dropwise to a mixture of 1 mol of 4-bromotoluene [106-38-7] and 1.2 mol of aluminium(III) chloride at room temperature. The reaction mixture is heated at 50° C. for 5 hours. It is then poured into ice. It is extracted with dichloromethane (2×)—the combined organic phases are washed successively with water, 2% NaOH and again with water, dried with sodium sulphate and evaporated. The title compound is identified by means of its Rf from the residue by flash chromatography (SiO₂ 60 F).

The following compound is prepared in an analogous manner to the process described in Example 73:
74 5-Amino-7-[4-ethyl-3-(4-methoxybutyryl)benzyl]-4-hydroxy-2-isopropyl-8-methylnonanoic acid (tetrahydropyran-4-yl)amide

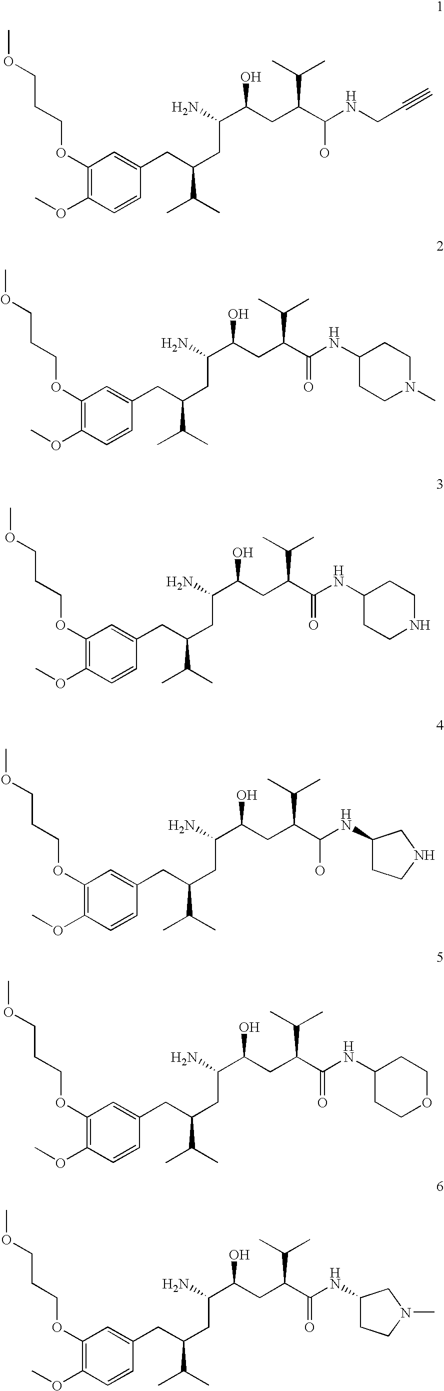

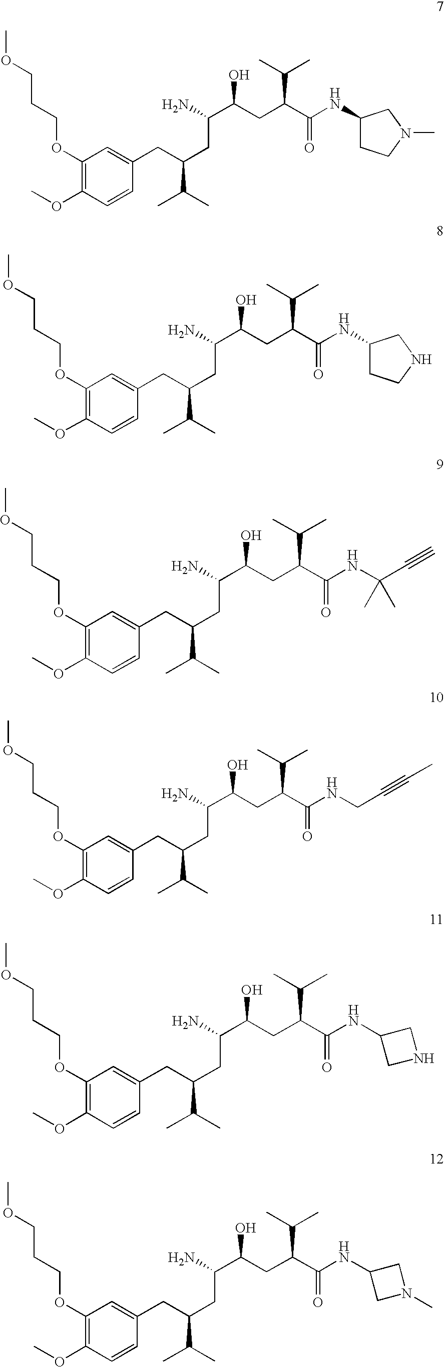

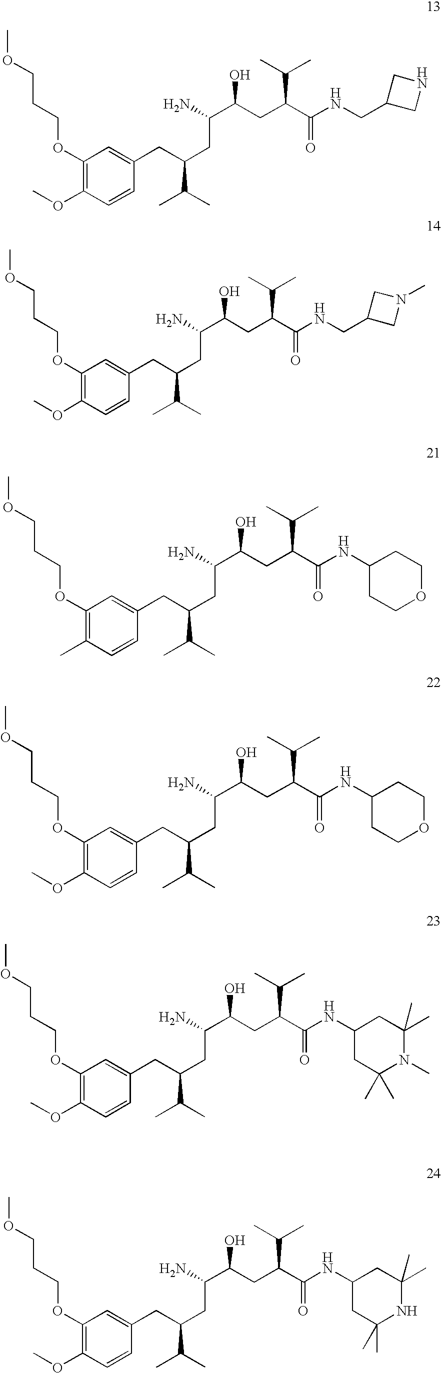

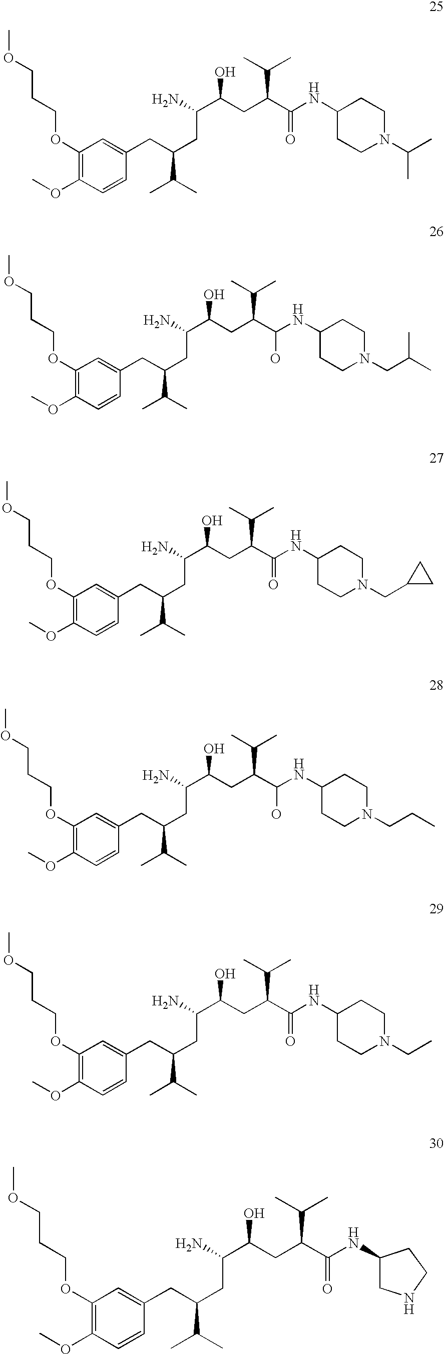

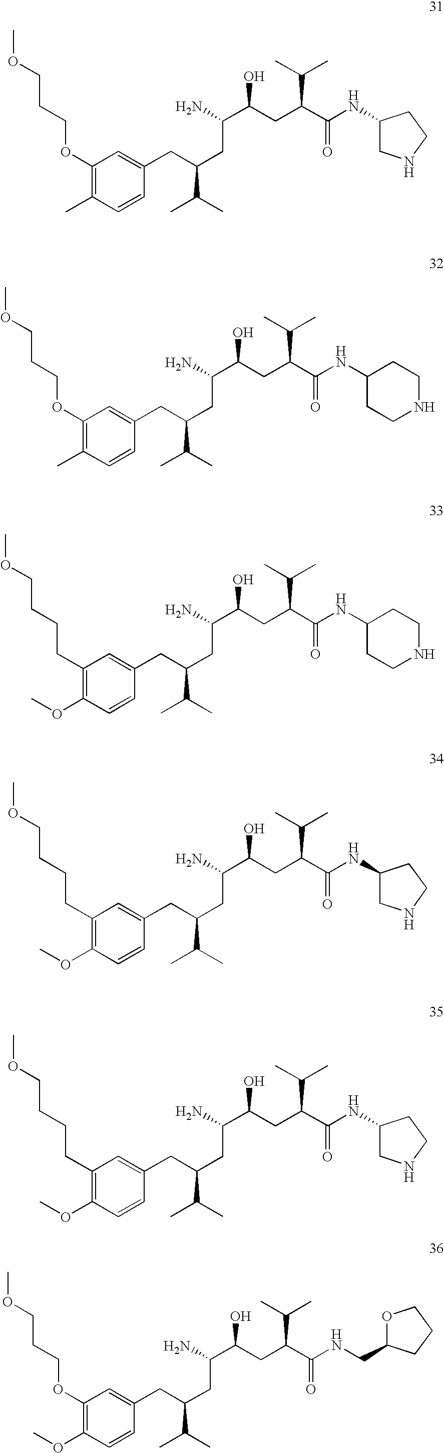

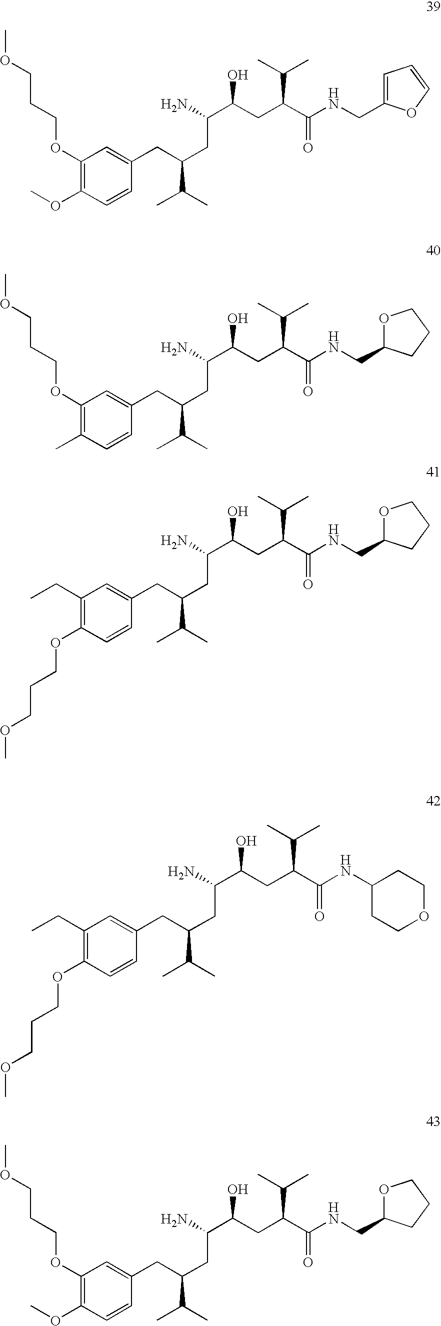

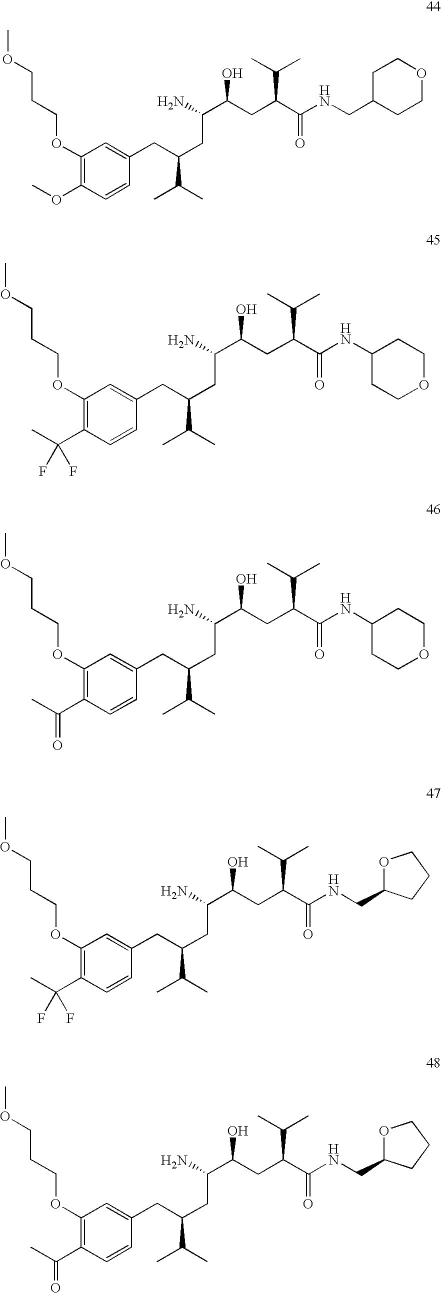

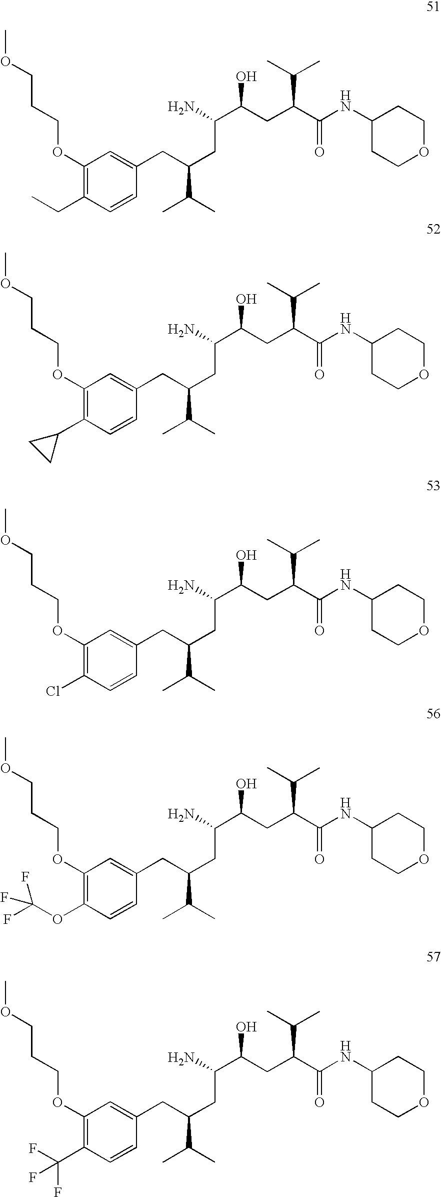

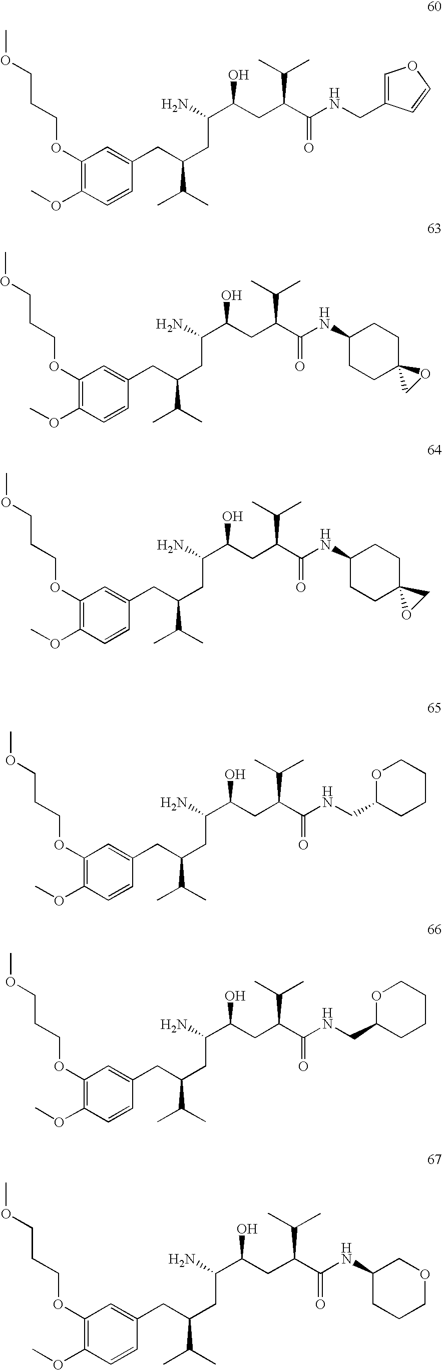

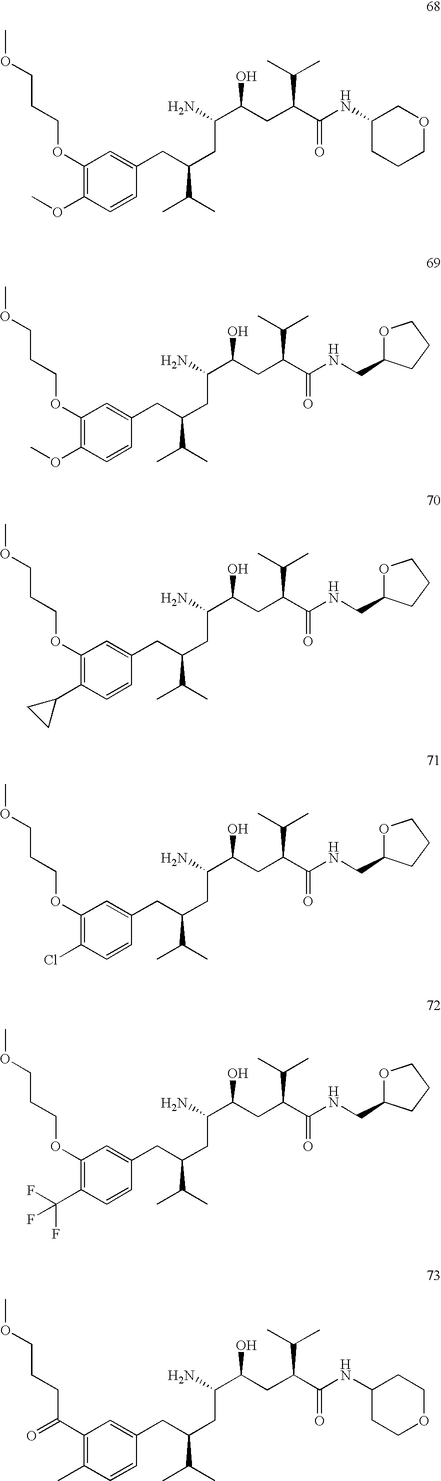

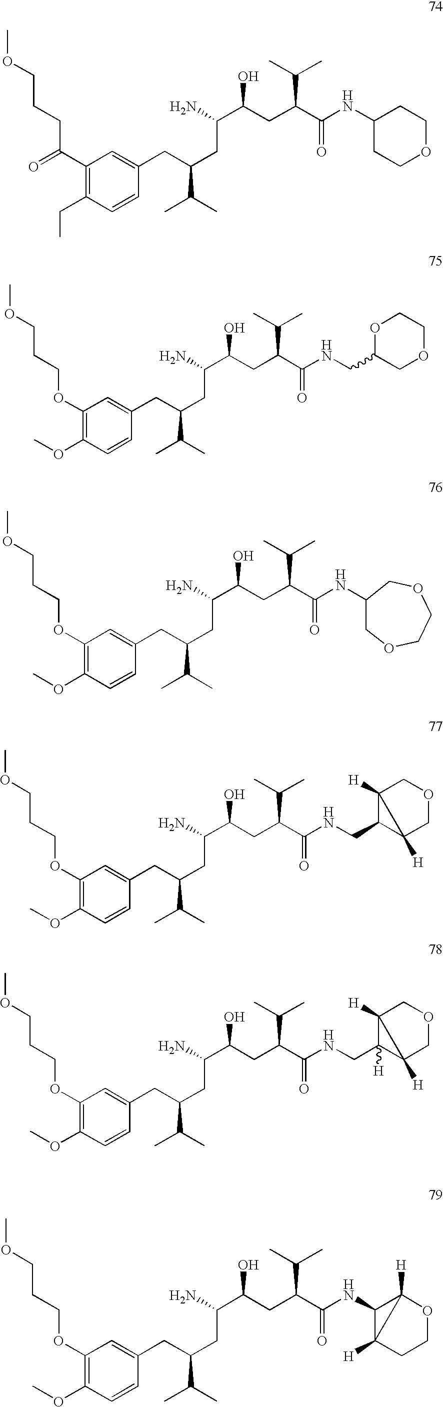

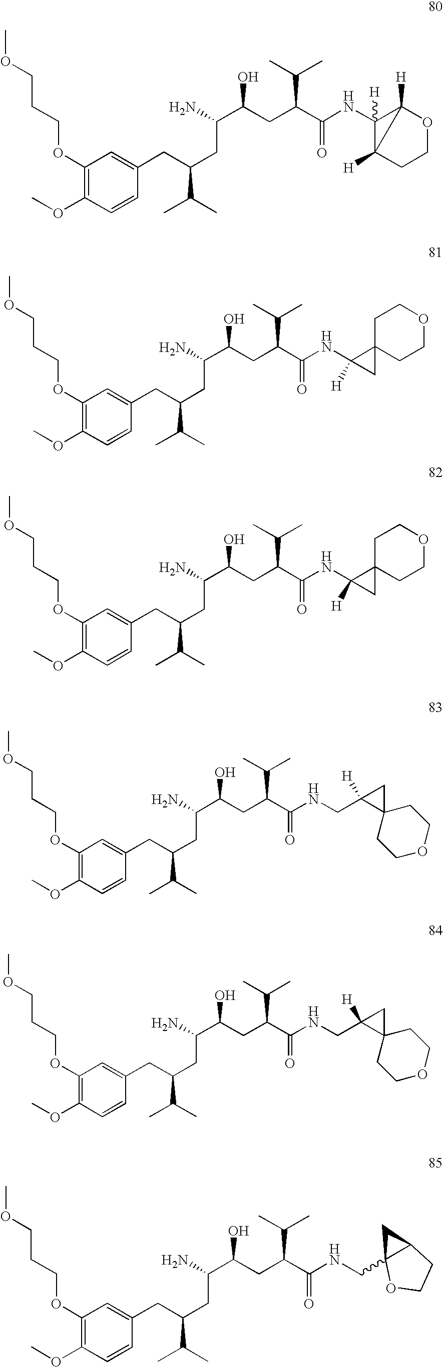

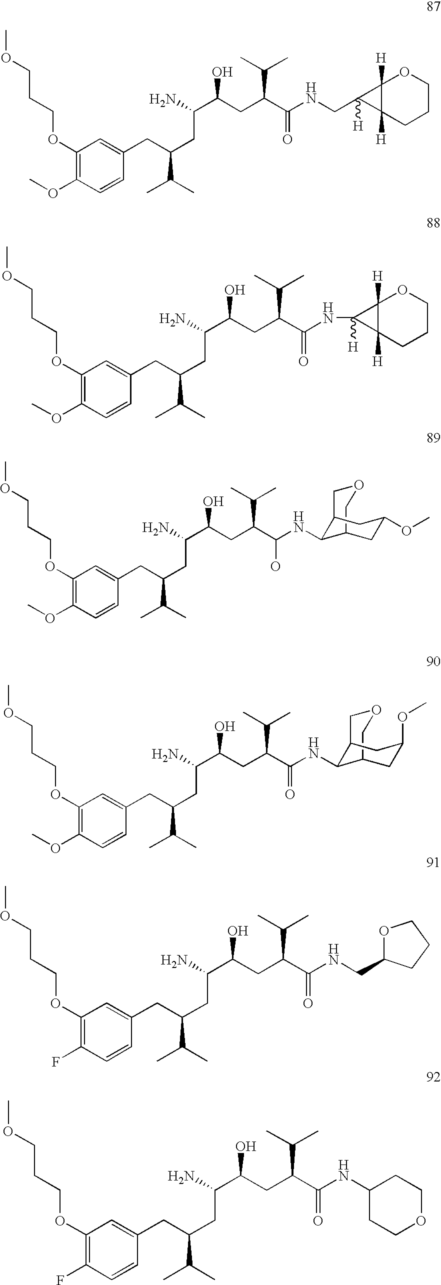

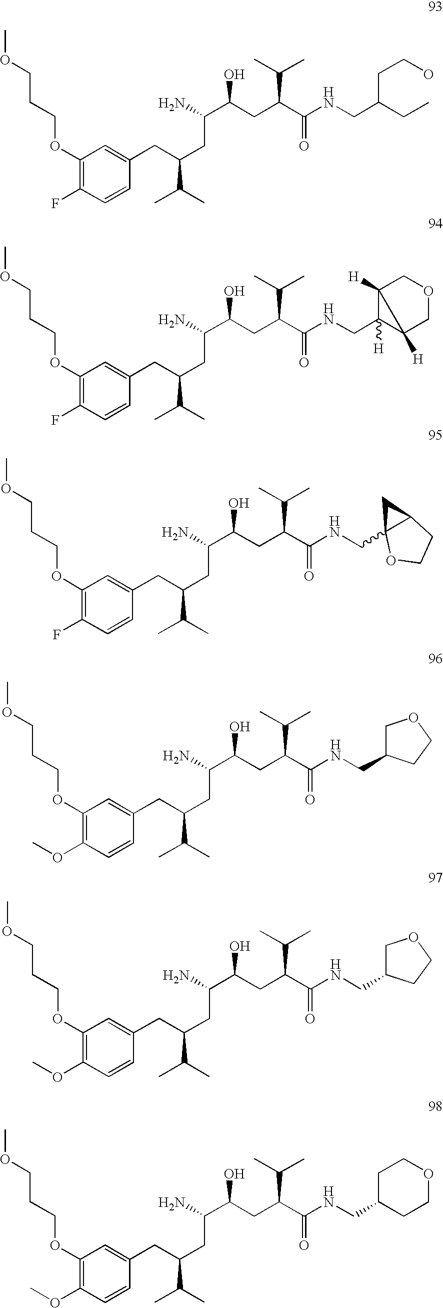

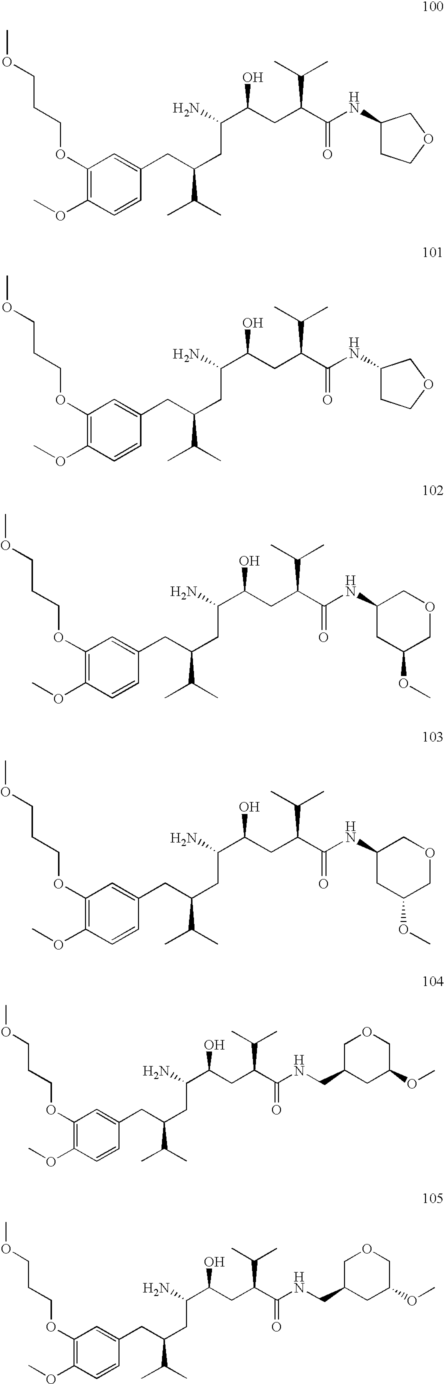

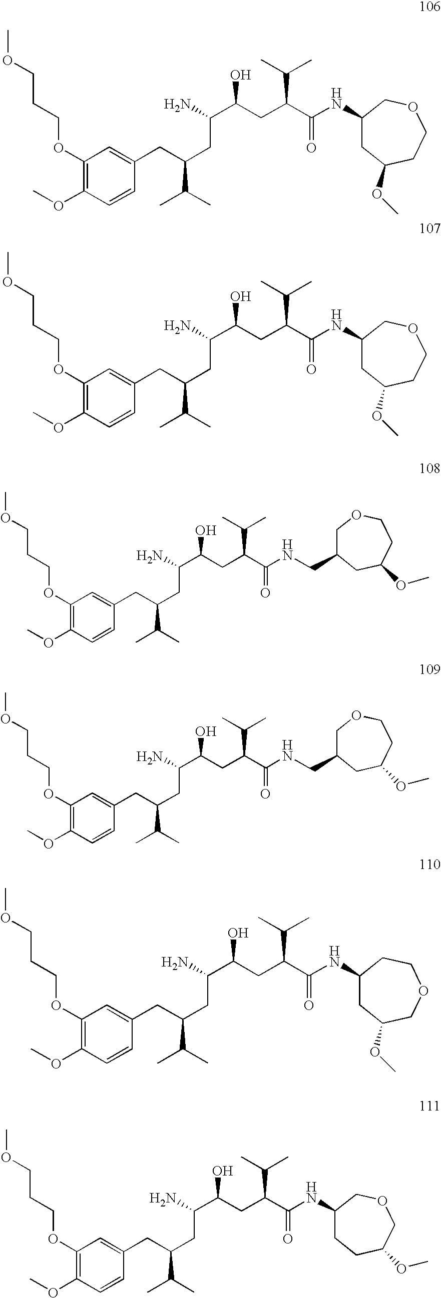

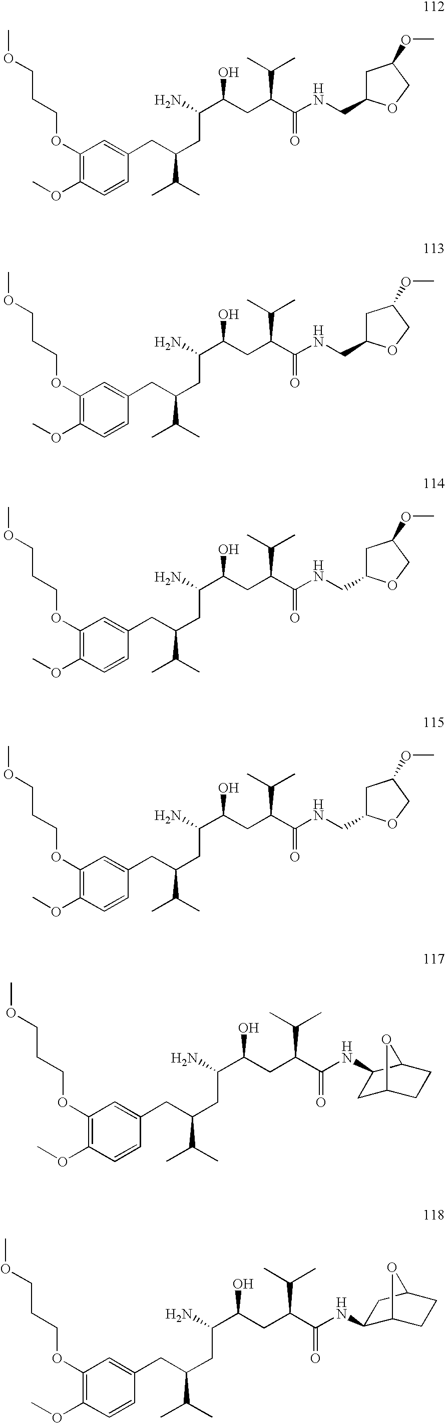

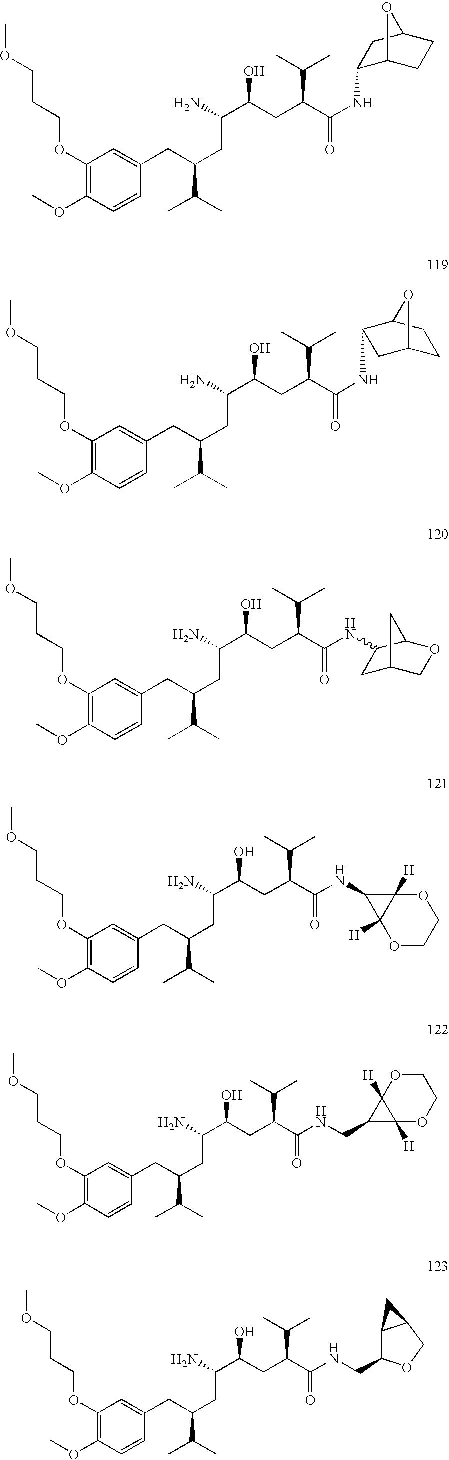

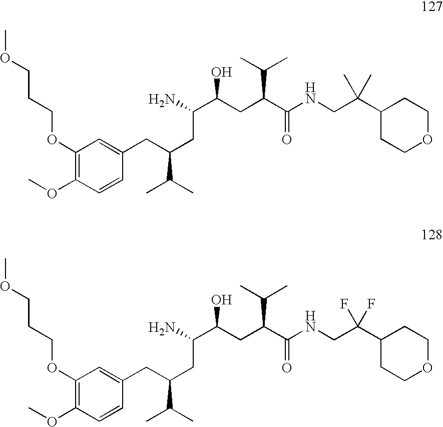

1-13. (canceled)

14. A compound of the formula 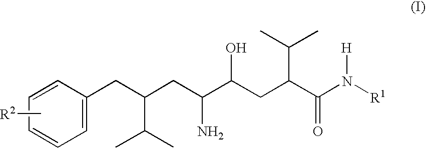
in which
R¹ is a) saturated heterocyclyl which is bonded via a C atom and where the heterocyclyl radical contains oxygen as heteroatom and which is optionally substituted one or more times by C₁₋₈alkanoyl, C₂₋₆alkenyl, C₂₋₆alkynyl, C₁₋₆alkoxy, C₁₋₆alkoxy-C₁₋₆alkoxy, C₁₋₆alkoxy-C₁₋₆alkyl, C₁₋₆alkoxycarbonylamino, C₁₋₆alkyl, C₀₋₆alkylcarbonylamino, C₁₋₆alkylcarbonyloxy, C₁₋₆alkylenedioxy, optionally N-mono or N,N-di-C₁₋₆-alkylated amino, aryl, optionally N-mono or N,N-di-C₁₋₆-alkylated carbamoyl, optionally esterified carboxy, cyano, C₃₋₈cycloalkoxy, C₃₋₈cycloalkyl-C₀₋₆alkyl, halogen, halo-C₁₋₆alkoxy, halo-C₁₋₆alkyl, heteroaryl, unsaturated, partially saturated or saturated heterocyclyl, hydroxy, nitro or oxo; or
b) dioxanyl-, dioxepanyl-, dioxolanyl-, oxepanyl-, tetrahydropyranyl-, tetrahydrofuranyl-, or bicyclic saturated heterocyclyl-C₁₋₄alkyl and where the heterocyclyl radical contains oxygen as heteroatom, each of which is optionally substituted one or more times by C₁₋₈alkanoyl, C₂₋₆alkenyl, C₂₋₆alkynyl, C₁₋₆alkoxy, C₁₋₆alkoxy-C₁₋₆alkoxy, C₁₋₆alkoxy-C₁₋₆alkyl, C₁₋₆alkoxycarbonylamino, C₁₋₆alkyl, C₀₋₆alkylcarbonylamino, C₁₋₆alkylcarbonyloxy, C₁₋₆alkylenedioxy, optionally N-mono or N,N-di-C₁₋₆-alkylated amino, aryl, optionally N-mono or N,N-di-C₁₋₆-alkylated carbamoyl, optionally esterified carboxy, cyano, C₃₋₈cycloalkoxy, C₃₋₈cycloalkyl-C₀₋₆alkyl, halogen, halo-C₁₋₆alkoxy, halo-C₁₋₆alkyl, heteroaryl, unsaturated, partially saturated or saturated heterocyclyl, hydroxy, nitro or oxo; or
c) C₂₋₈alkynyl which is optionally substituted one or more times by C₁₋₈alkanoyl, C₂₋₆alkenyl, C₂₋₆alkynyl, C₁₋₆alkoxy, C₁₋₆alkoxy-C₁₋₆alkoxy, C₁₋₆alkoxy-C₁₋₆alkyl, C₁₋₆alkoxycarbonylamino, C₁₋₆alkyl, C₀₋₆alkylcarbonylamino, C₁₋₆alkylcarbonyloxy, C₁₋₆alkylenedioxy, optionally N-mono or N,N-di-C₁₋₆-alkylated amino, aryl, optionally N-mono or N,N-di-C₁₋₆-alkylated carbamoyl, optionally esterified carboxy, cyano, C₃₋₈cycloalkoxy, C₃₋₈cycloalkyl-C₀₋₆alkyl, halogen, halo-C₁₋₆-alkoxy, halo-C₁₋₆alkyl, heteroaryl, unsaturated, partially saturated or saturated heterocyclyl, hydroxy, nitro or oxo; or
R² is, independently of one another, 1-4 radicals selected from:
C₁₋₈alkanoyl, C₁₋₈alkanoyl-C₂₋₄alkoxy bearing the alkanoyl group in a position higher than α, C₁₋₈alkanoylamino-C₁₋₄alkoxy, N—C₁₋₄alkanoylamino-C₁₋₄alkyl, C₁₋₈alkanoyloxy-C₁₋₄alkyl, N′—C₂₋₈alkanoyIpiperazino-C₁₋₄alkoxy, N′—C₂₋₈alkanoyIpiperazino-C₄alkyl, C₁₋₈alkanesulphonyl-C₁₋₄alkoxy, C₁₋₈alkanesulphonyl-C₁₋₄alkyl, C₁₋₈alkanesulphonylamino-C₁₋₄alkoxy, C₁₋₄alkanesulphonylamino-C₁₋₄alkyl, C₁₋₈alkanesulphonyl-C₁₋₄(hydroxy)alkoxy, C₂₋₈alkenyloxy, C₂₋₈alkenyloxy-C₁₋₄alkoxy, C₂₋₈alkenyloxy-C₁₋₄alkyl, C₁₋₈alkoxy, C₁₋₆alkoxy-C₁₋₆alkanoyl, C₁₋₄alkoxy-C₂₋₄alkenyl, C₁₋₄alkoxy-C₂₋₄alkenyloxy, C₁₋₄alkoxy-C₁₋₄alkoxy, C₁₋₄alkoxy-C₁₋₄alkoxy-C₁₋₄alkyl, C₁₋₄alkoxy-C₁₋₄alkyl, C₁₋₄alkoxycarbonyl-C₁₋₄alkoxy, C₁₋₄alkoxycarbonyl-C₁₋₄alkyl, C₁₋₈alkoxycarbonylamino-C₁₋₄alkoxy, C₁₋₈alkoxycarbonylamino-C₁₋₄alkyl, C₁₋₈alkyl, C₁₋₄alkylamino, N,N-di-C₁₋₄alkylamino, C₁₋₄alkylamino-C₁₋₄alkoxy, N,N-di-C₁₋₄alkylamino-C₁₋₄alkoxy, C₁₋₄alkylamino-C₁₋₄alkyl, N,N-di-C₁₋₄alkylamino-C₁₋₄alkyl, N-mono- or N,N-di-C₁₋₄alkylcarbamoyl-C₁₋₄alkoxy, N-mono- or N,N-di-C₁₋₄alkylcarbamoyl-C₁₋₄alkyl, N′—C₁₋₄alkylpiperazino-C₁₋₄alkoxy,N′—C₁₋₄alkylpiperazino-C₁₋₄alkyl, C₁₋₄alkylthio-C₁₋₄alkoxy, C₁₋₄alkylthio-C₁₋₄(hydroxy)alkoxy, C₁₋₄alkylthio-C₁₋₄alkyl, amino-C₂₋₄alkoxy, amino-C₁₋₄alkyl, carbamoyl-C₁₋₄alkoxy, carbamoyl-C₁₋₈alkyl, carboxy-C₁₋₄alkoxy, carboxy-C₁₋₄alkyl, cyano-C₁₋₄alkoxy, cyano-C₁₋₄alkyl, C₃₋₈-cycloalkoxy, C₃₋₈cycloalkoxy-C₁₋₄alkoxy, C₃₋₈cycloalkoxy-C₁₋₄alkyl, C₃₋₈cycloalkyl, S,S-dioxothiomorpholino-C₁₋₄alkoxy, S,S-dioxothiomorpholino-C₁₋₄alkyl, halogen, halo-C₁₋₄alkoxy, halo-C₁₋₄alkyl, halo-C₂₋₈(hydroxy)alkoxy, hydroxy, hydroxy-C₂₋₈alkoxy, hydroxy-C₂₋₈alkyl, imidazolylthio-C₁₋₄alkoxy, imidazolylthio-C₁₋₄alkyl, optionally N-oxidized morpholino-C₁₋₄alkoxy, morpholino-C₁₋₄alkyl, S-oxothiomorpholino-C₁₋₄alkoxy, S-oxothiomorpholino-C₁₋₄alkyl, piperazino-C₁₋₄alkoxy, piperazino-C₁₋₄alkyl, piperidino-C₁₋₄alkoxy, piperidino-C₁₋₄alkyl, optionally partially hydrogenated pyridyl- or N-oxidopyridyl-C₁₋₄alkoxy, optionally partially hydrogenated pyridyl- or N-oxidopyridyl-C₁₋₄alkyl, optionally N-oxidized pyridylthio-C₁₋₄alkoxy, optionally N-oxidized pyridylthio-C₁₋₄alkyl, pyrimidinylthio-C₁₋₄alkoxy, pyrimidinylthio-C₁₋₄alkyl, pyrrolidino-C₁₋₄alkoxy, pyrrolidino-C₁₋₄alkyl, thiazolinylthio-C₁₋₄alkoxy, thiazolinylthio-C₁₋₄alkyl, thiazolyl-C₁₋₄alkoxy, thiazolylthio-C₁₋₄alkoxy, thiazolylthio-C₁₋₄alkyl, thiomorpholino-C₁₋₄alkoxy, thiomorpholino-C₁₋₄alkyl, trifluoro-C₁₋₈alkanesulphonyl-C₁₋₄alkoxy, trifluoro-C₁₋₈alkanesulphonylamino-C₁₋₄alkyl, phenyl or naphthyl which is unsubstituted or mono-, di- or trisubstituted by C₁₋₄alkoxy, C₁₋₄alkyl, C₁₋₄alkylamino, di-C₁₋₄alkylamino, halogen, hydroxy and/or trifluoromethyl, and phenyl- or naphthyl-C₁₋₄alkoxy which is unsubstituted or mono-, di- or trisubstituted by C₁₋₄alkoxy, C₁₋₄alkyl, C₁₋₄alkylamino, di-C₁₋₄alkylamino, halogen, hydroxy and/or trifluoromethyl, and its salt or compound in which one or more atoms are replaced by their stable, non-radioactive isotopes, especially pharmaceutically acceptable salt.

15. A compound according to claim 14 of the formula 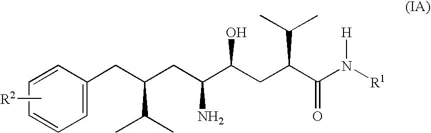
in which R¹ and R² has the meanings according to claim 14.

16. A compound according to claim 14, of the formula 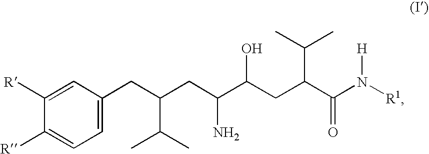
in which
R¹ has the meaning indicated for the compound of the formula (I) and
R′ and R″, independently of one another, have the meanings indicated for R² for the compound of the formula (I).

17. A compound according to claim 14, in which
R¹ is dioxanyl-, [1,4]dioxepanyl-, dioxolanyl-, oxepanyl-, tetrahydropyranyl-, tetrahydrofuranyl-, 2,5-dioxabicyclo[4.1.0]heptanyl-, 2-oxabicyclo[2.2.1]heptanyl-, 2-oxabicyclo[4.1.0]heptanyl-, 3-oxabicyclo[4.1.0]heptanyl-, 7-oxabicyclo[2.2.1]heptanyl-, 2-oxabicyclo[3.1.0]hexanyl-, 3-oxabicyclo[3.1.0]hexanyl-, 1-oxaspiro[2.5]octanyl-, 6-oxaspiro[2.5]octanyl- or 3-oxabicyclo[3.3.1]nonanyl-C₀₋₄alkyl, each of which is optionally substituted one or more times by C₁₋₈alkanoyl, C₂₋₆alkenyl, C₂₋₆alkynyl, C₁₋₆alkoxy, C₁₋₆alkoxy-C₁₋₆alkoxy, C₁₋₆alkoxy-C₁₋₆alkyl, C₁₋₆alkoxycarbonylamino, C₁₋₆alkyl, C₀₋₆alkylcarbonylamino, C₁₋₆alkylcarbonyloxy, C₁₋₆alkylenedioxy, optionally N-mono or N,N-di-C₁₋₆-alkylated amino, aryl, optionally N-mono or N,N-di-C₁₋₆-alkylated carbamoyl, optionally esterified carboxy, cyano, C₃₋₈cycloalkoxy, C₃₋₈cycloalkyl-C₀₋₆alkyl, halogen, halo-C₁₋₆alkoxy, halo-C₁₋₆alkyl, heteroaryl, unsaturated, partially saturated or saturated heterocyclyl, hydroxy, nitro or oxo.

18. A compound according to claim 14, in which
R² is, independently of one another, 1-4 radicals selected from: C₁₋₈alkanoylamino-C₁₋₄alkoxy, N—C₁₋₄alkanoylamino-C₁₋₄alkyl, C₁₋₈alkoxy, C₁₋₄alkoxy-C₁₋₄alkoxy, C₁₋₄alkoxy-C₁₋₄alkoxy-C₁₋₄alkyl, C₁₋₄alkoxy-C₁₋₄alkyl, C₁₋₈alkoxycarbonylamino-C₁₋₄alkoxy, C₁₋₈alkoxycarbonylamino-C₁₋₄alkyl, C₁₋₈alkyl, halogen, trifluoromethoxy and trifluoromethyl.

19. A compound according to claim 14, in which
R¹ is optionally substituted C₂₋₆alkynyl, optionally substituted saturated bicyclic heterocyclyl-C₀₋₄alkyl, optionally substituted pyrrolidinyl, C₁₋₆-alkylated or C₃₋₈cycloalkyl-C₀₋₆-alkylated piperidine, optionally substituted tetrahydrofuranyl, optionally substituted tetrahydrofuranylmethyl, optionally substituted tetrahydropyranyl or optionally substituted tetrahydropyranylmethyl.

20. A compound according to claim 14, in which
R¹ is optionally substituted tetrahydrofuranylmethyl or optionally substituted tetrahydropyranylmethyl.

21. A compound according to claim 15, in which
R′ is C₁₋₄alkoxy-C₁₋₄alkoxy;
R″ is fluorine; and
R¹ is optionally substituted tetrahydrofuranylmethyl, optionally substituted tetrahydropyranylmethyl, optionally substituted saturated heterocyclyl which is bonded via a C atom or optionally substituted saturated bicyclic heterocyclyl-C₀₋₄alkyl, where the heterocyclyl radical in each case comprises an oxygen atom as heteroatom.

22. The use of a compound of the general formula (I) according to claim 14 for producing a medicine.

23. The use of a compound of the general formula (I) according to claim 14 for producing a human medicine for preventing, for delaying the progression or for treating high blood pressure, heart failure, glaucoma, myocardial infarction, renal failure, restenoses or stroke.

24. A method for preventing, for delaying the progression or for treating high blood pressure, heart failure, glaucoma, myocardial infarction, renal failure, restenoses or stroke, where a therapeutically effective amount of a compound of the general formula (I) according to claim 14 is used.

25. A pharmaceutical product comprising a compound of the general formula (I) according to claim 14, and usual excipients.

26. A pharmaceutical combination in the form of a product or of a kit composed of individual components consisting a) of a compound of the general formula (I) according to claim 14, and b) at least one pharmaceutical form whose active ingredient has a cardiovascular effect.
